(19) United States
(12) Patent Application Publication
(10) Pub. No.: US 2019/0224164 A1
(43) Pub. Date: Jul. 25, 2019

(54) COMPOSITIONS AND METHODS FOR TREATMENT OF COPD

(71) Applicant: Bodor Laboratories, Inc., Miami, FL (US)

(72) Inventor: Nicholas S. BODOR, Bal Harbour, FL (US)

(21) Appl. No.: 16/312,995

(22) PCT Filed: Jun. 30, 2017
(86) PCT No.: PCT/US2017/040348

§ 371 Date: Dec. 21, 2018

Related U.S. Application Data
(60) Provisional application No. 62/357,711, filed on Jul. 1, 2016.
(60) Provisional application No. 62/357,730, filed on Jul. 1, 2016.

(51) Int. Cl. A61K 31/40; A61P 11/06; A61P 37/08; A61K 31/573; A61K 45/06; A61K 9/00

(52) U.S. Cl. CPC A61K 31/40; A61P 11/06; A61P 37/08; A61K 9/008; A61K 45/06; A61K 9/0043; A61K 31/573

(57) ABSTRACT

Methods and compositions for treating chronic obstructive pulmonary disease (COPD) and other obstructive diseases of the respiratory tract by administering soft anticholinergic esters once or twice daily with fewer systemic side-effects than glycopyrrolate.

### CROSS-REFERENCE TO RELATED APPLICATIONS

Priority is claimed to U.S. Provisional Patent Application Nos. 62/357,711, filed on Jul. 1, 2016 and 62/357,730, filed on Jul. 1, 2016. Each of the Provisional Patent Applications are hereby incorporated by reference in their entirety. A right is hereby reserved to have patentability determinations made on the basis of the applicable sections of Public Law 112-29.

### FIELD

The present disclosure relates to anticholinergic compounds and formulations and their use.

### BACKGROUND

Various anticholinergic compounds and formulations for those compounds have been previously described. Muscarinic receptor antagonists are frequently used therapeutic agents that inhibit the effects of acetylcholine by blocking its binding to muscarinic cholinergic receptors at neuroeffector sites on smooth muscle, cardiac muscle, and gland cells as well as in peripheral ganglia and in the central nervous system (CNS). However, their side effects, which can include dry mouth, photophobia, blurred vision, urinary hesitancy and retention, drowsiness, dizziness, restlessness, irritability, disorientation, hallucinations, tachycardia and cardiac arrhythmias, nausea, constipation, and severe allergic reactions, often limit their clinical use. However, currently used locally active anticholinergics can exhibit unwanted systemic side effects which can limit the dosage that can be safely administered.

Glycopyrrolate is among the quaternary ammonium anticholinergics which have reduced CNS-related side effects as they cannot cross the blood-brain barrier; however, because glycopyrrolate is eliminated mainly as unchanged drug or active metabolite, its topical and other local administration is often associated with common undesirable anticholinergic systemic side effects. To increase the therapeutic index of anticholinergics, the soft drug approach has been applied in a number of different designs starting from various lead compounds, but there is a need for optimizing soft anticholinergics with clinically meaningful biological activity. These muscarinic antagonists, just as all other soft drugs, are designed to elicit their intended pharmacological effect at the site of application, but to be quickly metabolized into their designed-in, inactive metabolite upon entering the systemic circulation and to be rapidly eliminated from the body, resulting in reduced systemic side effects and an increased therapeutic index.

Soft anticholinergic zwitterions have been described in US Patent Publication No. 2012/0141401 (now U.S. Pat. No. 8,568,699), and its related patents, U.S. Pat. Nos. 8,071,639; 7,538,219; and 7,417,147. Soft anticholinergic esters have been described in US Patent Publication No. 2012/0177590 (now U.S. Pat. No. 8,628,759) and its related patents U.S. Pat. Nos. 8,147,809; 7,576,210; and 7,399,861. Although these published applications and patents identified the potential for the zwitterion or ester forms of anticholinergics to be used for treating various conditions, the fact that activity and duration of action against COPD (chronic obstructive pulmonary disease) and other obstructive diseases of the respiratory tract are unexpectedly high herein, based on a comparison to published mydriasis data, was not known or previously described or suggested. Indeed, the quick metabolism and rapid elimination described for the soft anticholinergic esters in the art militate against their being useful for once or twice a day respiratory therapy. Likewise, the fact that the zwitterions are described in the art as having only about one-tenth the anticholinergic activity of the esters makes the zwitterions unlikely to be useful for once or twice daily respiratory therapy.

Each of the U.S. Patent Publications Nos. 2012/0141401 (U.S. Pat. No. 8,568,699) and 2012/0177590 (U.S. Pat. No. 8,628,759), and the related patents U.S. Pat. Nos. 8,147,809; 8,071,693; 7,576,210; 7,538,219; 7,417,147; and 7,399,861 are hereby incorporated by reference in their entireties and relied upon.

The soft anticholinergic esters of U.S. Pat. Nos. 8,628,729; 8,147,809; 7,576,210; and 7,399,861 have been previously proposed for use in a variety of pharmaceutical forms for various conditions requiring use of a local active, but not systemically active, anticholinergic agent. That is, these anticholinergics, which are soft drugs are designed to elicit their desired pharmacological effect at the site of application, but to be quickly metabolized into their designed-in, relatively inactive metabolite upon entering the systemic circulation, affording reduced side-effects. Indeed, these soft anticholinergic agents were previously proposed for use in the treatment of congestive obstructive pulmonary disease (COPD) and their obstructive disease of the respiratory tract. Nevertheless, it was expected that rapid inactivation to the much less active metabolite would prevent practical use as infrequently as once or twice per day. This is due to the fact that the esters were found to be very short-acting in mydriatic studies. In the eye, the unique alkyl ester function of these compounds was rapidly hydrolyzed and deactivated, such that a mydriatic activity was very short term. It was expected that the unique alkyl ester function of the soft esters would also be rapidly hydrolyzed by esterases, particularly cholinestearase, and deactivated in the respiratory system, especially the lungs, so that these compounds would not be practical for use in a once or twice a day regime or regimen, for example once a day in the treatment of COPD.

The soft anticholinergic zwitterions of U.S. Pat. Nos. 8,568,699; 8,071,639; 7,538,219; and 7,417,147 have been previously proposed for use in a variety of pharmaceutical forms for various conditions requiring use of a local active, but not systemically active, anticholinergic agent. That is, these anticholinergics have been among compounds previously described as soft anticholinergic zwitterions which are products of the hydrolysis of the corresponding soft anticholinergic esters. See, Bodor U.S. Pat. No. 8,147,809 and other U.S. and foreign counterparts thereof. See also, Wu et al., “Pharmacokinetic and Pharmacodynamic Evaluations of the Zwitterionic Metabolite of a New Series of N-Substituted Soft Anticholinergics”, Pharmaceutical Research, Vol. 22, No. 12, pp. 2035-2044, 12 Dec. 2005 (available online 26 Sep. 2005), Kluwer Academic, Plenum Publishers, U.S. The above-mentioned patent documents describe the synthesis and resolution of representative zwitterions and also contain pharmacological test data.

According to the patents and applications describing the soft anticholinergic zwitterions, the compounds of this type are much less active anticholinergics than the corresponding esters, by about an order of magnitude, that is, by a factor of 10, yet are nevertheless useful as anticholinergics; among their anticholinergic uses, the compounds of this type are taught to be useful in treating overactive bladder, COPD and other respiratory conditions, and also in inducing short-acting mydriasis and thus can be used to dilate the pupils of the eyes in vision testing.

Mydriatic studies in rabbit eyes have been previously described for an exemplary zwitterion, compared to glycopyrrolate, tropicamide and two soft anticholinergic esters. The zwitterion produced local mydriatic activity after topical administration but only with a short duration of action. The racemic form showed even lower potency.

Furthermore, the exemplary zwitterion has been found to not cause any observable irritation reactions, such as eye-closing, lacrimation, or mucous discharge; and unlike conventional anticholinergics, it did not cause pupil dilation in the contralateral, untreated eye, indicating not only low topical and systemic side effects, but also rapid elimination from the systemic circulation.

The fact is that the zwitterions were found to be very short-acting in mydriatic studies and were known as very weak in terms of intrinsic anticholinergic activity as characterized by pA2 (as reported earlier). In addition, when administered intravenously, the zwitterions were eliminated very quickly. Thus, the “inactive metabolite” (decrease of at least 10 fold in pA2 compared to the corresponding soft esters) was not considered to be sufficiently active or long-acting for the once daily or twice daily treatment of obstructive diseases of the respiratory tract, for example COPD and asthma.

### SUMMARY

The subject application concerns methods and pharmaceutical formulations for treating an obstructive disease of the respiratory tract (e.g. COPD) in subjects, in particular, in humans, suffering from said obstructive disease. A pharmaceutical composition for use herein comprises at least one soft anticholinergic agent, which is a soft ester analog of glycopyrrolate as described in more detail below, in a sufficient or an effective amount or concentration that reduces or inhibits at least one symptom of said obstructive disease, especially an amount that improves breathing. One embodiment is a pharmaceutical composition comprising: (a) at least one compound having the formula (1):

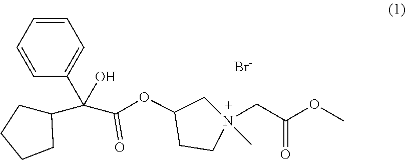

wherein R is C₁-C₈ straight or branched chain alkyl, said compound having the R, S, or RS stereoisomeric configuration at the 2 position and 1′ and 3′ positions, or a stereoisomeric mixture thereof, in an amount sufficient to reduce or inhibit at least one symptom of said obstructive disease; and (b) at least one pharmaceutically acceptable carrier or excipient, formulated for administration once daily or twice daily by oral inhalation or nasally. When R is methyl or ethyl, the formulation is preferably anhydrous.

A preferred embodiment of a pharmaceutical composition for use herein comprises: (a) at least one compound having the following stereospecific formula (2):

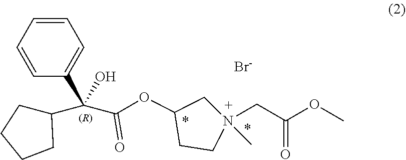

wherein R is C₁-C₈ straight or branched chain alkyl, said compound having the R stereoisomeric configuration at the 2 position and having the R, S, or RS stereoisomeric configuration at the 1′ and 3′ positions (designated by asterisks), or a stereoisomeric mixture thereof, and (b) at least one pharmaceutically acceptable carrier or excipient, formulated for administration once daily or twice daily by oral inhalation or nasally. When R is methyl or ethyl, the formulation is preferably anhydrous. The amount of compound of formula (2) is sufficient to inhibit or decrease at least one symptom of the obstructive respiratory disease.

Methods of treating an obstructive disease of the respiratory tract (e.g. COPD), or inhibiting or ameliorating or decreasing or reducing symptoms thereof, by using at least one compound of formula (1) or (2) above or a pharmaceutical composition as described herein comprising at least one such compound, are also included. The methods comprise, for example, administering nasally (to the nasal mucosa) or by oral inhalation (to the bronchial tubes and/or lungs) of a subject suffering from an obstructive disease of the respiratory tract, once or twice daily, at least one compound of formula (1) or (2) or a pharmaceutical composition comprising at least one compound of formula (1) or (2) above and at least one non-toxic pharmaceutically acceptable carrier or excipient, in an amount of compound of formula (1) or (2) sufficient to reduce or inhibit at least one symptom of said obstructive disease. The composition is formulated according to the particular obstructive disease to be treated. Formulation for oral inhalation (which can also be termed “pulmonary inhalation”) is contemplated for treatment of COPD, asthma, bronchitis, bronchiectasis, acute lung injury, acute respiratory distress syndrome (ARDS) and cystic fibrosis. Formulation for nasal administration is acceptable for treating allergic rhinitis or infectious rhinitis, except in severe cases where oral inhalation is preferred.

In an alternative embodiment, the pharmaceutical composition is a combination product comprising: (i) at least one at least one soft ester analog of glycopyrrolate, as described in more detail below, in a sufficient or an effective amount or concentration that reduces or inhibits at least one symptom of said obstructive disease, especially an amount that improves breathing; and (ii) at least one zwitterion, as described in more detail below, in an amount sufficient to prolong activity in reducing or inhibiting said at least one symptom. One embodiment is a pharmaceutical composition comprising: (a) at least one ester compound having the formula (1):

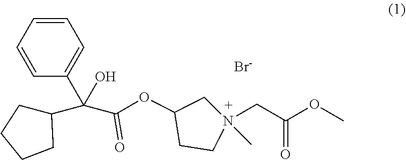

wherein R is C₁-C₈ straight or branched chain alkyl, said compound having the R, S, or RS stereoisomeric configuration at the 2 position and 1′ and 3′ positions, or a stereoisomeric mixture thereof, in an amount sufficient to reduce or inhibit at least one symptom of said obstructive disease; (b) at least one non-toxic pharmaceutically acceptable carrier or excipient, formulated for administration once daily or twice daily by oral inhalation or nasally; and (c) at least one zwitterion selected from the group consisting of:
(i) (±) 3-(2-cyclopentyl-2-phenyl-2-hydroxyacetoxy)-1 (carboxymethyl) 1-methylpyrrolidinium inner salt;
(ii) (2R) 3-(2-cyclopentyl-2-phenyl-2-hydroxyacetoxy)-1-(carboxymethyl)-1-methylpyrrolidinium inner salt;
(iii) (2R, 1′R, 3′R) 3-(2-cyclopentyl-2-phenyl-2-hydroxyacetoxy)-1-(carboxymethyl)-1-methylpyrrolidinium inner salt;
(iv) (2R, 1'S, 3′R) 3-(2-cyclopentyl-2-phenyl-2-hydroxyacetoxy)-1-(carboxymethyl)-1-methylpyrrolidinium inner salt;
(v) (2R, 1′R, 3'S) 3-(2-cyclopentyl-2-phenyl-2-hydroxyacetoxy)-1-(carboxymethyl)-1-methylpyrrolidinium inner salt;
(vi) (2R, 1'S, 3'S) 3-(2-cyclopentyl-2-phenyl-2-hydroxyacetoxy)-1-(carboxymethyl)-1-methylpyrrolidinium inner salt;
(vii) (2R, 3′R) 3-(2-cyclopentyl-2-phenyl-2-hydroxyacetoxy)-1-(carboxymethyl)-1-methylpyrrolidinium inner salt;
(viii) (2R, 3'S) 3-(2-cyclopentyl-2-phenyl-2-hydroxyacetoxy)-1-(carboxymethyl)-1-methylpyrrolidinium inner salt;
(ix) (2R, I′R) 3-(2-cyclopentyl-2-phenyl-2-hydroxyacetoxy)-1-(carboxymethyl)-1-methylpyrrolidinium inner salt;
(x) (2R, 1 'S) 3-(2-cyclopentyl-2-phenyl-2-hydroxyacetoxy)-1-(carboxymethyl)-1-methylpyrrolidinium inner salt; and
   a mixture of said at least one zwitterion with at least one stereoisomer thereof, in an amount or concentration sufficient to prolong activity in reducing or inhibiting said at least one symptom; and optionally, (c) at least one non-toxic pharmaceutically acceptable carrier or excipient, formulated for administration once daily or twice daily by oral inhalation or nasally. When R is methyl or ethyl, the formulation is preferably anhydrous.

A preferred embodiment of a pharmaceutical composition or combination product for use herein comprises: (a) at least one ester compound having the following stereospecific formula (2):

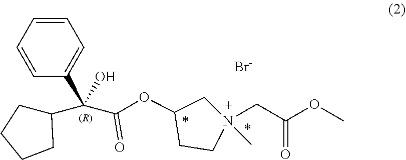

wherein R is C₁-C₈ straight or branched chain alkyl, said compound having the R stereoisomeric configuration at the 2 position and having the R, S, or RS stereoisomeric configuration at the 1′ and 3′ positions (designated by asterisks), or a stereoisomeric mixture thereof; (b) at least one zwitterion selected from the group consisting of:
(i) (±) 3-(2-cyclopentyl-2-phenyl-2-hydroxyacetoxy)-1-(carboxymethyl)-1-methylpyrrolidinium inner salt;
(ii) (2R) 3-(2-cyclopentyl-2-phenyl-2-hydroxyacetoxy)-1-(carboxymethyl)-1-methylpyrrolidinium inner salt;
(iii) (2R, 1′R, 3′R) 3-(2-cyclopentyl-2-phenyl-2-hydroxyacetoxy)-1-(carboxymethyl)-1-methylpyrrolidinium inner salt;
(iv) (2R, 1'S, 3′R) 3-(2-cyclopentyl-2-phenyl-2-hydroxyacetoxy)-1-(carboxymethyl)-1-methylpyrrolidinium inner salt;
(v) (2R, 1′R, 3'S) 3-(2-cyclopentyl-2-phenyl-2-hydroxyacetoxy)-1-(carboxymethyl)-1-methylpyrrolidinium inner salt;
(vi) (2R, 1'S, 3'S) 3-(2-cyclopentyl-2-phenyl-2-hydroxyacetoxy)-1-(carboxymethyl)-1-methylpyrrolidinium inner salt;
(vii) (2R, 3′R) 3-(2-cyclopentyl-2-phenyl-2-hydroxyacetoxy)-1-(carboxymethyl)-1-methylpyrrolidinium inner salt;
(viii) (2R, 3'S) 3-(2-cyclopentyl-2-phenyl-2-hydroxyacetoxy)-1-(carboxymethyl)-1-methylpyrrolidinium inner salt;
(ix) (2R, I′R) 3-(2-cyclopentyl-2-phenyl-2-hydroxyacetoxy)-1-(carboxymethyl)-1-methylpyrrolidinium inner salt;
(x) (2R, 1'S) 3-(2-cyclopentyl-2-phenyl-2-hydroxyacetoxy)-1-(carboxymethyl)-1-methylpyrrolidinium inner salt; and
   a mixture of said at least one zwitterion with at least one stereoisomer thereof, and optionally, (c) at least one non-toxic pharmaceutically acceptable carrier or excipient, formulated for administration once daily or twice daily by oral inhalation or nasally.

When R is methyl or ethyl, the formulation is preferably anhydrous. The amount of ester compound of formula (2) is sufficient to inhibit or decrease at least one symptom of the obstructive respiratory disease. The amount of zwitterion is also as defined with formula (1) above.

Methods of treating an obstructive disease of the respiratory tract (e.g. COPD), or inhibiting or ameliorating or decreasing or reducing symptoms thereof, by using at least one compound of formula (1) or (2) above or a pharmaceutical composition as described herein comprising at least one such compound, are also included. The methods comprise, for example, administering nasally (to the nasal mucosa) or by oral inhalation (to the bronchial tubes and/or lungs) of a subject suffering from an obstructive disease of the respiratory tract, once or twice daily, at least one compound of formula (1) or (2) or a pharmaceutical composition comprising at least one compound of formula (1) or (2) above and at least one non-toxic pharmaceutically acceptable carrier or excipient, in an amount of compound of formula (1) or (2) sufficient to reduce or inhibit at least one symptom of said obstructive disease. The composition is formulated according to the particular obstructive disease to be treated. Formulation for oral inhalation (which can also be termed “pulmonary inhalation”) is contemplated for treatment of COPD, asthma, bronchitis, bronchiectasis, acute lung injury, acute respiratory distress syndrome (ARDS) and cystic fibrosis. Formulation for nasal administration is acceptable for treating allergic rhinitis or infectious rhinitis, except in severe cases where oral inhalation is preferred.

Despite the foregoing, while an anhydrous form is appropriate for storage stability, especially when R is methyl or ethyl, it is not necessary that the pharmaceutical composition be anhydrous at the time of administration.

Advantageously, the method can surprisingly provide reduction of at least one symptom of an obstructive disease of the respiratory tract, as compared to baseline conditions, for about 12 to 24 hours by an amount which is substantially equivalent to the reduction of said at least one symptom resulting from administration of a composition comprising about the same amount of glycopyrrolate, also compared to baseline conditions as that of ester compound of formula (1) or (2). Based on mydriatic studies, the soft esters were previously believed to hydrolyze too rapidly to provide substantially equivalent activity to glycopyrrolate over a prolonged period of up to 24 hours. The soft esters also have now been found to provide substantially equivalent results to glycopyrrolate while causing fewer systemic side-effects than glycopyrrolate. Thus, once a day treatment of COPD and other obstructive respiratory conditions with these soft esters is now possible. Moreover, although the zwitterion is much less active than the ester (by a factor of 10), it has different and stronger binding than the ester and is therefore able to prolong the activity of the soft ester in treating obstructive diseases of the respiratory tract. Thus, the use of ester compound and zwitterion together is particularly and surprisingly advantageous here.

### DETAILED DESCRIPTION

Throughout this specification and claims, the following definitions, general statements and illustrations are applicable.

The patents, published applications and scientific literature referred to herein establish the knowledge of those with skill in the art and are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compounds or compositions described herein, i.e., the process or composition is limited to the specified steps or materials and those which do not materially affect the basic and novel characteristics of the process or composition.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001).

As used herein, “treating” means reducing, hindering or inhibiting the development of, or controlling, inhibiting, alleviating and/or reversing one or more symptoms in the individual to which a compound of formula (1) or (2) together with a zwitterion (i)-(x), or a composition comprising a compound of formula (1) or (2) and zwitterion (i)-(x), has been administered, as compared to the symptoms of an individual not being administered the compound or composition. A practitioner will appreciate that the combinations, compositions, dosage forms and methods described herein are to be used in concomitance with continuous clinical evaluations by a skilled practitioner (physician or veterinarian) to determine subsequent therapy. Such evaluation will aid and inform in evaluating whether to increase, reduce or continue a particular treatment dose, and/or to alter the mode of administration.

The subject compounds or compositions can also prevent at least one of the symptoms, or prevent the occurrence of at least one of the symptoms, in the individual to which a composition comprising an ester compound of formula (1) or (2) and, optionally, the zwitterion (i)-(x) above has been administered, as compared to at least one of the symptoms of an individual not being administered the ester compound and, optionally, the zwitterion, or the composition. This is not a prevention of an obstructive disease of the respiratory tract in the absolute sense; it does not prevent the medical condition, rather it inhibits one or more manifestations of the condition for the period of time (hours) for which the administered dose is effective.

The methods described herein are intended for use with any subject/patient that may experience their benefits. Thus, the terms “subjects” as well as “patients,” “individuals” and “warm-blooded animals” and “mammals” include humans as well as non-human subjects, such as non-human animals that may experience the same or similar obstructive diseases of the respiratory tract.

The expression “an obstructive disease of the respiratory tract” encompasses any disease which interferes with breathing by obstructing the bronchi and/or lungs. Such obstructive diseases include asthma, bronchitis, COPD (chronic obstructive pulmonary disease), allergic rhinitis, infectious rhinitis, bronchiectasis, acute lung injury (ALI), acute respiratory distress syndrome (ARDS) and cystic fibrosis. For purposes of this disclosure, allergic and infectious rhinitis are referred to as less serious obstructive diseases and all of the other named diseases as serious or more serious diseases.

“Asthma” refers to a chronic lung disease causing bronchoconstriction (narrowing of the airways) due to inflammation (swelling) and tightening of the muscles around the airways. The inflammation also causes an increase in mucus production, which causes coughing that may continue for extended periods. Asthma is generally characterized by recurrent episodes of breathlessness, wheezing, coughing, and chest tightness, termed exacerbations. The severity of exacerbations can range from mild to life threatening. The exacerbations can be a result of exposure to e.g. respiratory infections, dust, mold, pollen, cold air, exercise, stress, tobacco smoke, and air pollutants.

“Bronchitis” refers to inflammation of the bronchi. The bronchi are airways of large and medium size. The symptoms of bronchitis include coughing (including coughing up mucus), shortness of breath, wheezing and chest discomfort. Bronchitis can be acute or chronic. Chronic bronchitis is frequently associated with COPD.

“COPD” refers to chronic obstructive pulmonary disease, primarily but not necessarily associated with past and present cigarette smoking. It involves airflow obstruction, mainly associated with emphysema and chronic bronchitis. Emphysema causes irreversible lung damage by weakening and breaking the air sacs within the lungs. Chronic bronchitis is an inflammatory disease, which increases mucus in the airways and bacterial infections in the bronchial tubes, resulting in obstructed airflow.

“Allergic rhinitis” refers to acute rhinitis or nasal rhinitis, including hay fever. It is caused by allergens such as pollen or dust. It may produce sneezing, congestion, runny nose, and itchiness in the nose, throat, eyes, and ears.

“Infectious rhinitis” refers to acute rhinitis or nasal rhinitis of infectious origin. It is caused by upper respiratory tract infection by infectious rhinoviruses, coronaviruses, influenza viruses, parainfluenza viruses, respiratory syncytical virus, adenoviruses, coxsackieviruses, echoviruses, or Group a beta-hemolytic Streptococci and is generically referred to as the common cold. It may produce sneezing, congestion, runny nose, and itchiness in the nose, throat, eyes, and ears.

“Bronchiectasis” refers to an obstructive lung disease of the airways, or bronchi, in which parts of the airways become permanently enlarged and scarred. Symptoms usually include a mucus-producing chronic cough. Other symptoms include shortness of breath. The disease often leads to frequent lung infections. Causes include tuberculosis, pneumonia, problems of the immune system and cystic fibrosis. Nearly all cases of cystic fibrosis ultimately lead to severe bronchiectasis.

“Cystic fibrosis”, also known as mucoviscidosis or CF, is a genetic disorder that mainly affects the lungs. Symptoms include coughing up mucus and difficulty breathing as a result of frequent lung infections. Cardiorespiratory complications are the most common cause of death, although the disease also affects the pancreas, liver, kidneys and intestines.

“Acute lung injury” (ALI) is a condition diagnosed clinically and radiologically based on the presence of non-cardiogenic pulmonary edema and respiratory failure in a critically ill patient. ALI encompasses a continuum of radiographic and clinical changes, acute respiratory distress syndrome (ARDS) being the severe end of the continuum.

“Acute respiratory distress syndrome” (ARDS) is a disease of the alveoli, which are the microscopic air sacs of the lungs, leading to decreased exchange of oxygen and carbon dioxide. Pathological changes associated with ARDS include release of inflammatory chemicals, breakdown of the cells lining the lungs' blood vessels, surfactant loss, fluid accumulation in the lungs and excessive scarring. Diffuse alveolar damage (DAD) is characteristic.

Compounds useful in the methods and compositions herein include those of the formula (1):

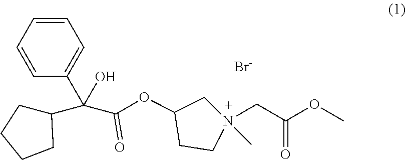

wherein R is C₁-C₈ straight or branched chain and the compound has the R, S, or RS stereoisomeric configuration at the 2 position and at the 1′ and 3′ positions, or a stereoisomeric mixture thereof.

Compounds having the R configuration with respect to chiral center 2 are of particular interest for use in the instant methods and compositions. For example, a preferred compound useful in a method or composition herein has the stereospecific formula

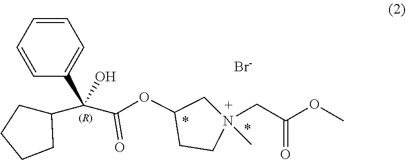

wherein R is C₁-C₈ straight or branched chain alkyl, said compound having the R stereoisomeric configuration at the 2 position and the R, S, or RS stereoisomeric configuration at the 1′ and 3′ positions (designated by asterisks), or a stereoisomeric mixture thereof.

The moiety R in formulas (1) and (2) can be methyl, ethyl, n-propyl, n-butyl, n-pentyl, n-hexyl, n-heptyl or n-octyl or their branched chain isomere.

In the compounds of formulas (1) and (2), R is preferably C₁-C₈ straight chain alkyl, more preferably methyl, ethyl, n-butyl, n-hexyl or n-octyl.

The compound of formula (2) preferably has the R or RS configuration at the 3′ position.

The compounds of formulas (1) and (2) wherein R is methyl or ethyl are of special interest, especially those of formula (2) having the R or RS configuration at the 3′ position.

The following compounds are of particular interest for use in a method or composition of the present description:
(1) 3-(2-cyclopentyl-2-phenyl-2-hydroxyacetoxy)-1-(methoxycarbonylmethyl)-1-methylpyrrolidinium bromide;
(2) 3-(2-cyclopentyl-2-phenyl-2-hydroxyacetoxy)-1 (ethoxycarbonylmethyl)-1-methylpyrrolidinium bromide;
(3) (2R) 3-(2-cyclopentyl-2-phenyl-2-hydroxyacetoxy)-1-(methoxycarbonylmethyl)-1-methylpyrrolidinium bromide;
(4) (2R) 3-(2-cyclopentyl-2-phenyl-2-hydroxyacetoxy)-1-(ethoxycarbonylmethyl)-1-methylpyrrolidinium bromide;
(5) (2R, 3′R) 3-(2-cyclopentyl-2-phenyl-2-hydroxyacetoxy)-1-(methoxycarbonylmethyl)-1-methylpyrrolidinium bromide;
(6) (2R, 3'S) 3-(2-cyclopentyl-2-phenyl-2-hydroxyacetoxy)-1-(methoxycarbonylmethyl)-1-methylpyrrolidinium bromide;
(7) (2R, 3'S) 3-(2-cyclopentyl-2-phenyl-2-hydroxyacetoxy)-1-(ethoxycarbonylmethyl)-1-methylpyrrolidinium bromide;
(8) (2R, 3′R) 3-(2-cyclopentyl-2-phenyl-2-hydroxyacetoxy)-1-(ethoxycarbonylmethyl)-1-methylpyrrolidinium bromide;
(9) (2R,11R,31S) 3-(2-cyclopentyl-2-phenyl-2-hydroxyacetoxy)-1-(ethoxycarbonylmethyl)-1-methylpyrrolidinium bromide;
(10) (2R,11S,31S) 3-(2-cyclopentyl-2-phenyl-2-hydroxyacetoxy)-1-(ethoxycarbonylmethyl)-1-methylpyrrolidinium bromide;
(11) (2R, 1′R, 3′R) 3-(2-cyclopentyl-2-phenyl-2-hydroxyacetoxy)-1-(ethoxycarbonylmethyl)-1-methylpyrrolidinium bromide;
(12) (2R, 1'S, 3′R) 3-(2-cyclopentyl-2-phenyl-2-hydroxyacetoxy)-1-(ethoxycarbonylmethyl)-1-methylpyrrolidinium bromide;
(13) (2R, 1′R, 3'S) 3-(2-cyclopentyl-2-phenyl-2-hydroxyacetoxy)-1-(methoxycarbonylmethyl)-1-methylpyrrolidinium bromide;
(14) (2R, 1'S, 3'S) 3-(2-cyclopentyl-2-phenyl-2-hydroxyacetoxy)-1-(methoxycarbonylmethyl)-1-methylpyrrolidinium bromide;
(15) (2R, 1′R, 3′R) 3-(2-cyclopentyl-2-phenyl-2-hydroxyacetoxy)-1-(methoxycarbonylmethyl)-1-methylpyrrolidinium bromide;
(16) (2R, 1'S, 3′R) 3-(2-cyclopentyl-2-phenyl-2-hydroxyacetoxy)-1-(methoxycarbonylmethyl)-1-methylpyrrolidinium bromide;
(17) (2R, 1′R, 3'S) 3-(2-cyclopentyl-2-phenyl-2-hydroxyacetoxy)-1-(n-hexyloxycarbonylmethyl)-1-methylpyrrolidinium bromide;
(18) (2R, 1'S, 3'S) 3-(2-cyclopentyl-2-phenyl-2-hydroxyacetoxy)-1-(n-hexyloxycarbonylmethyl)-1-methylpyrrolidinium bromide;
(19) (2R, 1′R, 3′R) 3-(2-cyclopentyl-2-phenyl-2-hydroxyacetoxy)-1-(n-hexyloxycarbonylmethyl)-1-methylpyrrolidinium bromide;
(20) (2R, 1'S, 3′R) 3-(2-cyclopentyl-2-phenyl-2-hydroxyacetoxy)-1-(n-hexyloxycarbonylmethyl)-1-methylpyrrolidinium bromide.
(21) (2R, 1′R, 3'S) 3-(2-cyclopentyl-2-phenyl-2-hydroxyacetoxy)-1-methyl-1-(n-octyloxycarbonylmethyppyrrolidinium bromide;
(22) (2R, 1'S, 3'S) 3-(2-cyclopentyl-2-phenyl-2-hydroxyacetoxy)-1-methyl-1-(n-octyloxycarbonylmethyppyrrolidinium bromide;
(23) (2R, 1′R, 3′R) 3-(2-cyclopentyl-2-phenyl-2-hydroxyacetoxy)-1-methyl-1-(n-octyloxycarbonylmethylpyrrolidinium bromide;
(24) (2R, 1'S, 3′R) 3-(2-cyclopentyl-2-phenyl-2-hydroxyacetoxy)-1-methyl-1-(n-octyloxycarbonylmethyppyrrolidinium bromide;
(25) 3-(2-cyclopentyl-2-phenyl-2-hydroxyacetoxy)-1-(n-hexyloxycarboxymethyl)-1-methylpyrrolidinium bromide;
(26) (2R) 3-(2-cyclopentyl-2-phenyl-2-hydroxyacetoxy)-1-(n-hexyloxycarboxymethyl)-1-methylpyrrolidinium bromide;
(27) (2R, 3′R) 3-(2-cyclopentyl-2-phenyl-2-hydroxyacetoxy)-1-(n-hexyloxycarboxymethyl)-1-methylpyrrolidinium bromide;
(28) (2R, 3'S) 3-(2-cyclopentyl-2-phenyl-2-hydroxyacetoxy)-1-(n-hexyloxycarboxymethyl)-1-methylpyrrolidinium bromide;
(29) 3-(2-cyclopentyl-2-phenyl-2-hydroxyacetoxy)-1-(n-octyloxycarbonylmethyl)-1-methylpyrrolidinium bromide;
(30) (2R) 3-(2-cyclopentyl-2-phenyl-2-hydroxyacetoxy)-1-(n-octyloxycarbonylmethyl)-1-methylpyrrolidinium bromide;
(31) (2R, 3′R) 3-(2-cyclopentyl-2-phenyl-2-hydroxyacetoxy)-1-(n-octyloxycarbonylmethyl)-1-methylpyrrolidinium bromide;
(32) (2R, 3'S) 3-(2-cyclopentyl-2-phenyl-2-hydroxyacetoxy)-1-(n-octyloxycarbonylmethyl)-1-methylpyrrolidinium bromide;
(33) 3-(2-cyclopentyl-2-phenyl-2-hydroxyacetoxy)-1-(n-butoxycarbonylmethyl)-1-methylpyrrolidinium bromide;
(34) (2R) 3-(2-cyclopentyl-2-phenyl-2-hydroxyacetoxy)-1-(n-butoxycarbonylmethyl)-1-methylpyrrolidinium bromide;
(35) (2R, 3′R) 3-(2-cyclopentyl-2-phenyl-2-hydroxyacetoxy)-1-(n-butoxycarbonylmethyl)-1-methylpyrrolidinium bromide;
(36) (2R, 3'S) 3-(2-cyclopentyl-2-phenyl-2-hydroxyacetoxy)-1-(n-butoxycarbonylmethyl)-1-methylpyrrolidinium bromide;
(37) (2R, 1′R, 3'S) 3-(2-cyclopentyl-2-phenyl-2-hydroxyacetoxy)-1-(n-butoxycarbonylmethyl)-1-methylpyrrolidinium bromide;
(38) (2R, 1'S, 3'S) 3-(2-cyclopentyl-2-phenyl-2-hydroxyacetoxy)-1-(n-butoxycarbonylmethyl)-1-methylpyrrolidinium bromide;
(39) (2R, 1′R, 3′R) 3-(2-cyclopentyl-2-phenyl-2-hydroxyacetoxy)-1-(n-butoxycarbonylmethyl)-1-methylpyrrolidinium bromide; and
(40) (2R, 1'S, 3′R) 3-(2-cyclopentyl-2-phenyl-2-hydroxyacetoxy)-1-(n-butoxycarbonylmethyl)-1-methylpyrrolidinium bromide.

Of these compounds, particular mention can be made of:
(a) (2R) 3-(2-cyclopentyl-2-phenyl-2-hydroxyacetoxy)-1-(methoxycarbonylmethyl)-1-methylpyrrolidinium bromide;
(b) (2R) 3-(2-cyclopentyl-2-phenyl-2-hydroxyacetoxy)-1-(ethoxycarbonylmethyl)-1-methylpyrrolidinium bromide;
(c) (2R) 3-(2-cyclopentyl-2-phenyl-2-hydroxyacetoxy-1-(n-hexyloxycarbonylmethyl)-1-methylpyrrolidinium bromide;
(d) (2R) 3-(2-cyclopentyl-2-phenyl-2-hydroxyacetoxy)-1-(n-octyloxycarbonylmethyl)-1-methylpyrrolidinium bromide;
(e) (2R) 3-(2-cyclopentyl-2-phenyl-2-hydroxyacetoxy)-1-(n-butoxycarbonylmethyl)-1-methylpyrrolidium bromide;
(f) (2R, 3′R) 3-(2-cyclopentyl-2-phenyl-2-hydroxyacetoxy)-1-(methoxycarbonylmethyl)-1-methylpyrrolidinium bromide;
(g) (2R, 3′R) 3-(2-cyclopentyl-2-phenyl-2-hydroxyacetoxy)-1-(ethoxycarbonylmethyl)-1-methylpyrrolidinium bromide;
(h) (2R, 3′R) 3-(2-cyclopentyl-2-phenyl-2-hydroxyacetoxy)-1-(n-hexyloxycarbonylmethyl)-1-methylpyrrolidium bromide;
(i) (2R, 3′R) 3-(2-cyclopentyl-2-phenyl-2-hydroxyacetoxy)-1-(n-octyloxycarbonylmethyl)-1-methylpyrrolidinium bromide; and
(j) (2R, 3′R) 3-(2-cyclopentyl-2-phenyl-2-hydroxyacetoxy)-1-(n-butoxycarbonylmethyl)-1-methylpyrrolidinium bromide.

The compounds of formulas (1) and (2) can be prepared according to methods generally described in U.S. Pat. No. 8,628,729, 8,147,809, 7,576,210 and 7,399,861 and in the related scientific literature articles. Individual stereoisomers can be prepared from resolved starting materials or intermediates, or can be prepared from the corresponding racemic mixture of formulas (1) and (2).

The zwitterions for use in the instant methods and compositions together with at least one ester compound of formula (1) or (2) constitute at least one zwitterion selected from the group consisting of:
(i) (±) 3-(2-cyclopentyl-2-phenyl-2-hydroxyacetoxy)-1-(carboxymethyl)-1-methylpyrrolidinium inner salt;
(ii) (2R) 3-(2-cyclopentyl-2-phenyl-2-hydroxyacetoxy)-1-(carboxymethyl)-1-methylpyrrolidinium inner salt;
(iii) (2R, 1′R, 3′R) 3-(2-cyclopentyl-2-phenyl-2-hydroxyacetoxy)-1-(carboxymethyl)-1-methylpyrrolidinium inner salt;
(iv) (2R, 1'S, 3′R) 3-(2-cyclopentyl-2-phenyl-2-hydroxyacetoxy)-1-(carboxymethyl)-1-methylpyrrolidinium inner salt;
(v) (2R, 1′R, 3'S) 3-(2-cyclopentyl-2-phenyl-2-hydroxyacetoxy)-1-(carboxymethyl)-1-methylpyrrolidinium inner salt;
(vi) (2R, 1'S, 3'S) 3-(2-cyclopentyl-2-phenyl-2-hydroxyacetoxy)-1-(carboxymethyl)-1-methylpyrrolidinium inner salt;
(vii) (2R, 3′R) 3-(2-cyclopentyl-2-phenyl-2-hydroxyacetoxy)-1-(carboxymethyl)-1-methylpyrrolidinium inner salt;
(viii) (2R, 3'S) 3-(2-cyclopentyl-2-phenyl-2-hydroxyacetoxy)-1-(carboxymethyl)-1-methylpyrrolidinium inner salt;
(ix) (2R, I′R) 3-(2-cyclopentyl-2-phenyl-2-hydroxyacetoxy)-1-(carboxymethyl)-1-methylpyrrolidinium inner salt; and
(x) (2R, 1'S) 3-(2-cyclopentyl-2-phenyl-2-hydroxyacetoxy)-1-(carboxymethyl)-1-methylpyrrolidinium inner salt;
   as well as mixtures of the zwitterions with one or more of their stereoisomers.

Zwitterion (i) above has the following structural formula:

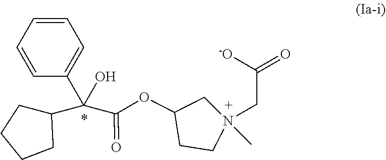

wherein the asterisk indicates that the compound is unresolved at the 2-position, that is, that the compound is a mixture of 2R and 2S stereoisomers. The compound is also unresolved at the 1′- and 3′-positions.

Zwitterion (ii) above can be represented by the following structural formula:

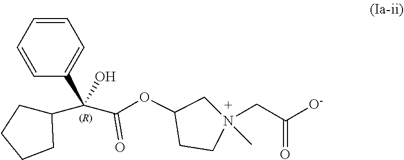

This compound has the 2R configuration, but is unresolved at the 1′ and 3′ positions.

Zwitterions (iii) through (x) have the structural formula:

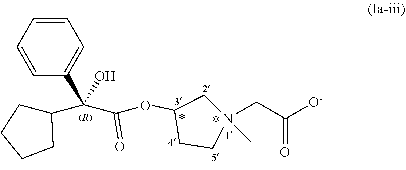

wherein the asterisks indicate that the compounds are resolved at one or both of the 1′ and 3′ positions. The zwitterion thus has one of the following configurations: (2R, 1′R, 3′R), (2R, 1′S, 3′R), (2R, 1′R, 3′S), (2R, 1′S, 3′S), (2R, 3′R), (2R, 3′S), (2R, 1′R) and (2R, 1′S); however, each zwitterion can be used in admixture with one or more of its stereoisomers, just as can the ester compounds of formula (1) or (2).

The above zwitterions (i)-(x) can be used alone or two or more of the above zwitterions can be used in combination with esters (1) or (2) in a single composition. Various methods of making the instant zwitterions are described in the art. A preferred zwitterion for use herein is compound (ii) above, also known as (2R)SGA or BOD-03. Another preferred zwitterion for use herein is compound (vii) above, also known as (2R,3′R)SGA or BOD-08. In a preferred embodiment, the stereoisomeric configuration at the 2,1′ and 3′ positions of the selected zwitterion matches the stereoisomeric configuration at the 2,1′ and 3′ positions, respectively in the ester of formula (1) or (2).

An anticholinergically effective amount of the ester component, either alone or as an ester/zwitterion combination, inhibits the effect of acetylcholine by blocking its binding to muscarinic cholinergic receptors at neuroeffector sites. Subjects in need of a method of eliciting an anticholinergic response are those suffering from conditions which respond to treatment with an anticholinergic agent, including subjects suffering from an obstructive disease of the respiratory tract, such as COPD and the other obstructive diseases of the respiratory tract identified herein.

The compound of formula (1) or (2) is typically administered in the form of a pharmaceutical composition comprising an anticholinergically effective amount of the compound and a non-toxic pharmaceutically acceptable (preferably anhydrous) carrier or excipient therefor. In alternative embodiments, the ester compound of formula (1) or (2) together with the zwitterion (i) through (x) is typically administered in the form of a pharmaceutical composition comprising an anticholinergically effective amount of the ester compound, an amount of the zwitterion (i)-(x) sufficient to prolong the activity of the ester in treating at least one symptom of the obstructive respiratory disorder, and a non-toxic pharmaceutically acceptable (preferably anhydrous) carrier or excipient thereof. Pharmaceutically acceptable carriers, or diluents, are well-known in the art. The carriers may be any inert material, organic or inorganic, powders, liquid, or gases suitable for administration by oral inhalation or nasally, such as: alcohol such as hexylene glycol, lactose (especially anhydrous lactose), magnesium stearate, isopropyl myristate, sorbitan trioleate, benzalkonium chloride, EDTA, monofluorotrichloromethane, difluorodichloromethane, HCl, 1,1,1,2-tetrafluoroethane, 1,1,1,2,3,3,3-heptafluoropropane and mixtures thereof. For storage stability over time, the ingredients, or at least those in contact with the compound of formula (1) or (2), should be anhydrous, particularly when R is methyl or ethyl.

It is apparent from the foregoing that compositions for use herein can contain conventional additives such as solvents, stabilizers, wetting agents, emulsifiers, buffers, binders, disintegrants, lubricants, glidants, antiadherents, propellants, and the like, just so long as the additives and compositions (or portions thereof in contact with the ester compound) are anhydrous, that is, free of water to the extent required to avoid significant negative impact on the storage stability of the compositions in which R in the compound of formula (1) or (2) is methyl or ethyl (by hydrolysis of the methyl or ethyl ester drug).

In preparing a formulation, it can be appropriate to mill one or both of the active compounds to provide the correct particle size prior to combining with the other ingredients. One or both of the active compounds can be milled to a particle size of less than 200 mesh.

For purposes of illustration, liquid formulation dosages are expressed based on a percent solution (g/100 ml or mg/100 ml) or percent concentration (w/v) unless otherwise stated. For inhalable powder formulation dosages, the percent concentration can be expressed as mg/mg, or w/w concentrations or μg per dosage unit or per capsule. For aerosols, % by weight is typically used unless otherwise stated. A person of ordinary skill in the art would readily understand the percent concentration in the context of the type of formulation described.

In general, a therapeutically effective or anticholinergically effective amount of a compound of formula (1) or (2) herein is an amount sufficient to significantly diminish or inhibit one or more symptoms of an obstructive disease of the respiratory tract, in particular, to ease breathing difficulties (by improving the condition of the airways and/or lungs). In adult patients, this amount is generally from about 20 to about 150 μg, preferably from about 50 to about 100 μg, once or twice daily, preferably once daily, by oral inhalation. Said amount of ester compound of formula (1) or (2) and, optionally, an amount of zwitterion as defined above sufficient to strengthen binding and to prolong the activity of the compound of formula (1) or (2) (by virtue of the zwitterion's superior binding and ability to build up in concentration at receptor sites). Typically the amounts used are in a molar ratio of ester to zwitterion of from about 2.5:1 to about 5:1, e.g., from about 50-100 μg of ester with about 20 μg of zwitterion, when approximated as a weight:weight ratio. The exact dosage of an ester compound and a zwitterion in the instant composition can vary depending on potency, the mode and frequency of administration, the application area, the age and weight of the subject and the nature and severity of the condition to be treated. In general, the amount of compound of formula (1) or (2) used in the method herein is about the same, on a molar basis, as the amount of glycopyrrolate which produces about the same effect. Yet the compounds of formulas (1) and (2) accomplish this with fewer systemic side effects than glycopyrrolate.

Administration of composition or a combination as described herein can thus provide a substantially identical, similar or improved clinical response in a subject, as compared to administration of a composition or combination containing approximately the same concentration of glycopyrrolate. Further, the results of this discovery are surprising in view of previously published mydriatic studies which suggested that the subject compounds in a composition or combination were required to be present in a concentration from 5 times to 10 times the concentration of a glycopyrrolate composition exhibiting a similar or substantially identical clinical response in the once or twice daily treatment of COPD or other obstructive respiratory disease, in particular, by oral inhalation.

Furthermore, the results of this discovery are especially surprising for multiple reasons. First, the results are surprising because the soft alkyl esters were previously believed to hydrolyze too rapidly to be useful in the once or twice daily treatment of COPD by oral inhalation. Indeed, it was known that these esters have a half-life in plasma of about 10 minutes and it was believed that they would be hydrolyzed rapidly in the airways/lungs by the enzyme butyrylcholinesterase (BChE). It has now been unexpectedly found that these esters are not hydrolyzed by BChE but rather by Paraoxonase 1. Paraoxonase 1 has less activity in the lungs/airways than in the general circulatory system. Therefore, the esters of formula (1) and (2), surprisingly, are not rapidly hydrolyzed in the lungs and airways and can be successfully delivered by oral inhalation to act in the lungs and airways to treat COPD and other obstructive respiratory diseases as infrequently as once per day. The absence of a large amount of Paraoxonase 1 in the lungs/airways encourages a particularly long acting activity in treating COPD and the like. At the same time, when the ester drug does reach the bloodstream, the designed-in breakdown to non-toxic entities by hydrolysis there avoids or greatly minimizes the systemic side-effects which characterize anticholinergics such as glycopyrrolate.

Secondly, the results of this discovery are surprising in view of previously published mydriatic studies which suggested that the subject zwitterions in a composition were required to be present in a concentration greater than their parent esters and from 5 times to 10 times the concentration of a glycopyrrolate composition exhibiting a similar or substantially identical clinical response. In rabbits, the zwitterions were also found to be four times shorter acting than their parent esters and over sixteen times shorter acting than glycopyrrolate mydriatically. However, because the esters and zwitterions exert their action by different mechanisms, and because the zwitterions bind more strongly than the esters due to the zwitterions' highly charged state, it is believed that the zwitterions can act synergistically to enhance the activity of the soft esters. Strengthening the overall binding prolongs and strengthens activity and, because the concentration of the zwitterion builds up over time and acts over time more so than the ester, thus, using the zwitterion in combination with the soft esters can lead to a better and longer effect in the treatment of COPD than does use of a compound of formula (1) or (2) alone.

The amount of zwitterion used in the methods described herein and present in the instant combinations and compositions is an amount sufficient to prolong the activity of the ester compound in reducing or inhibiting at least one symptom of the obstructive disease of the respiratory tract. The amount of the zwitterion can also be referred to as a synergistically effective amount, that is, an amount sufficient to enhance the activity or duration of action, or both, of the soft ester. Typically, the ester compound and zwitterion are co-administered from a combination or composition comprising both of them.

The zwitterions, while themselves much less active as anticholinergics than the ester compounds, are most preferably administered herein as synergists in amounts lower than amounts of zwitterions considered therapeutically effective for inhibiting or reducing one or more symptoms of an obstructive disease of the respiratory tract. While not wishing to be bound by a particular theory or mechanism, it is believed that, in these less than therapeutically effective (i.e. sub-therapeutic) amounts, the zwitterions are able to enhance the anticholinergic activity and/or duration of action of the ester compounds because of the zwitterions' stronger binding due to their charged nature, as well as their strong specificity for the M3 receptors. Because the esters have much higher intrinsic activity, the esters bind first to the receptors, but when they are finished exerting their anticholinergic activity, they are replaced at the receptor sites by the zwitterions, which have increased in concentration in the interim and very strongly bond to the M3 receptors, providing longer term activity than possible with the esters alone.

Whatever the final dosage form, the readily hydrolyzable nature of the ester of formula (1) or (2) in which R is methyl or ethyl must be taken into account in the preparation of the dosage form. Care must be taken that the preparation method does not combine the drug with a water-containing ingredient which would hydrolyze the drug during preparation or storage of the dosage form.

The ester compounds of formulas (1) and (2) can be used on their own or combined with other active substances of formula (1) or (2) according to the description. The same is true of the zwitterions.

The ester compounds of formulas (1) or (2) and the zwitterions as defined herein can optionally also be combined with other pharmaceutically active substances. These include, in particular, betamimetics, antiallergic agents, and especially corticosteroids (also termed “anti-inflammatory steroids”, “anti-inflammatory corticosteroids” or simply “steroids”) and combinations of these active substances. The combinations with betamimetics, antiallergics and/or corticosteroids are of interest in the treatment of obstructive diseases of the respiratory tract, especially COPD or asthma or other serious obstructive respiratory disease. Accordingly, they are primarily intended for administration by oral inhalation, as powders or aerosols.

Examples of betamimetics which can be used in conjunction with the ester compounds of formulas (1) or (2) and the zwitterions defined herein include compounds selected from the group consisting of bambuterol, bitolterol, carbuterol, clenbuterol, fenoterol, formoterol, hexoprenaline, ibuterol, pirbuterol, procaterol, reproterol, salmeterol, sulfphonterol, terbutaline, tulobuterol, 4-hydroxy-7-[2-{[2-{[3-(2-phenylethoxy)propyl]sulfonyl}ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N, N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylaminolethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert-butylamino)ethanol and 1-(4-ethoxycarbonylamino-3-cyano-5-fluorophenyl)-2-(tert-butylamino)ethanol, optionally in the form of their racemates, their enantiomers, their diastereomers, as well as optionally their pharmacologically acceptable acid addition salts. It is particularly preferable to use, as betamimetics, active substances of this kind, combined with the ester compounds of formulas (1) or (2), and the zwitterions as defined herein, selected from among fenoterol, formoterol, salmeterol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N, N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylaminolethanol, optionally in the form of their racemates, their enantiomers, their diastereomers, as well as optionally their pharmacologically acceptable acid addition salts. Of the betamimetics mentioned above, the compounds formoterol and salmeterol, optionally in the form of their racemates, their enantiomers, their diastereomers, as well as optionally their pharmaceutically acceptable acid addition salts, are particularly important.

The acid addition salts of the betamimetics selected from the group consisting of the hydrochloride, hydrobromide, sulfate, phosphate, fumarate, methanesulfonate and xinafoate are preferred herein. In the case of salmeterol, the salts selected from the group consisting of the hydrochloride, sulfate and xinafoate are particularly preferred, especially the sulfates and xinafoates. In the case of formoterol, the salts selected from among the hydrochloride, sulfate and fumerate are particularly preferred, especially the hydrochloride and fumarate. Of outstanding importance is formoterol fumarate.

The corticosteroids which can optionally, and, indeed, preferably, be used in conjunction with the ester compounds of formulas (1) or (2) and zwitterions as defined herein, include compounds selected from the group consisting of flunisolide, beclomethasone, triamcinolone, budesonide, fluticasone, mometasone, circlesonide, rofleponide, GW 215864, KSR 592, ST-126, loteprednol etabonate, etiprednol dichloracetate and dexamethasone. The preferred corticosteroids are those selected from the group consisting of flunisolide, beclomethasone, triamcinolone, loteprednol etabonate, etiprednol dichloracetate, budesonide, fluticasone, mometasone, ciclesonide and dexamethasone, while budesonide, fluticasone, loteprednol etabonate, etiprednol dichloracetate, mometasone and ciclesonide, especially budesonide, fluticasone, loteprednol etabonate and etiprednol dichloracetate, are of particular importance. Any reference to steroids herein also includes a reference to pharmaceutically acceptable salts or derivatives which can be formed from the steroids. Examples of possible salts or derivatives include: sodium salts, sulfobenzoates, phosphates, isonicotinates, acetates, propionates, dihydrogen phosphates, palmitates, pivalates and furoates.

When the corticosteroid is loteprednol etabonate, it can be advantageously combined with an enhancing agent selected from the group consisting of:
(a) 11β,17α-dihydroxyandrost-4-en-3-one-17β-carboxylic acid (cortienic acid, or CA);
(b) 11β,17α-dihydroxyandrosta-1,4-dien-3-one-17β-carboxylic acid (Δ¹ cortienic acid or Δ¹-CA);
(c) methyl 11β, 17α-dihydroxyandrost-4-en-3-one-17β-carboxylate (cortienic acid methyl ester, or MeCA);
(d) ethyl 11β,17α-dihydroxyandrost-4-en-3-one-17β-carboxylate (cortienic acid ethyl ester, or EtCA);
(e) methyl 11β,17α-dihydroxyandrosta-1,4-dien-3-one-17β-carboxylate (Δ¹ cortienic acid methyl ester, or Δ¹-MeCA); and
(f) ethyl 11β, 17α-dihydroxyandrosta-1,4-dien-3-one-17β-carboxylate (Δ′ cortienic acid ethyl ester, or Δ¹-EtCA),
   wherein the mole ratio of loteprednol etabonate to enhancing agent is from about 5:1 to about 0.5:1. Such combinations with these inactive metabolites are described in detail in WO 2005/000317 A1, incorporated by reference herein in its entirety and relied upon.

Examples of antiallergic agents which can be used as (i) a combination with the ester compounds of formula (1) or (2) and, optionally, (ii) as a combination with the ester compounds of formula (1) or (2) along with the zwitterions as defined herein include epinastin, cetirizin, azelastin, fexofenadin, levocabastin, loratadine, mizolastin, ketotifen, emedastin, dimetinden, clemastine, bamipin, cexchloropheniramine, pheniramine, doxylamine, chlorophenoxamine, dimenhydrinate, diphenhydramine, promethazine, ebastin, desloratidine and meclizine. Preferred antiallergic agents which can be used in combination with the ester compounds of formulas (1) or (2) and, optionally, with the zwitterions as defined herein are selected from the group consisting of epinastin, cetirizin, azelastin, fexofenadin, levocabastin, loratadine, ebastin, desloratidine and mizolastin, among which epinastin and desloratidine are particularly preferred. Any reference to the abovementioned antiallergic agents also includes a reference to any pharmacologically acceptable acid addition salts thereof which exist.

When the ester compounds of formulas (1) or (2) and combinations thereof with the zwitterions as defined herein are used in conjunction with other active substances, the combination with steroids or betamimetics, and most especially with steroids, is particularly preferred of the various categories of additional compounds mentioned above.

Whether or not the ester compounds of formulas (1) or (2) as defined herein are used in conjunction with other active substances as described above, they are typically administered in the form of a pharmaceutical composition comprising: (a) an amount of an ester compound of formula (1) or (2) sufficient or effective to reduce or inhibit at least one symptom of an obstructive respiratory disease; (b) a non-toxic pharmaceutically acceptable carrier or excipient therefor; and, optionally, (c) an amount of a zwitterion (i)-(x) as defined herein sufficient to prolong the activity of the ester compound in treating at least one symptom of the obstructive respiratory disorder. Pharmaceutically acceptable carriers, or diluents, are well-known in the art. The carriers may be any inert material, organic or inorganic, suitable for the desired route of administration and suitable for combination with the selected active compounds. Such compositions can also contain other pharmaceutically active agents, as noted above, and/or conventional additives such as stabilizers, buffers, binders, propellants, and the like.

The compounds of formulas (1) and (2), or combination thereof, can be brought into suitable dosage forms, that is, compositions for administration through the nasal or pulmonary route (typically via oral inhalation) in accordance with accepted pharmaceutical procedures. The route of administration and thus the dosage form will be chosen in light of the particular condition to be treated with the instant anticholinergic agents. Thus, when the compound of formula (1) or (2), or combination thereof, is administered to treat COPD or asthma, or other serious obstructive disease of the respiratory tract, the compounds can be advantageously administered via inhalation or insufflation; for such purposes, the compounds or combination thereof are advantageously in the form of an aerosol or a powder for inhalation. When administered to treat less serious respiratory disorders such as rhinitis (allergic or infectious), a nasal spray, mist or gel can be advantageous.

For purposes of illustration, dosages are expressed based on the inhalation of an aerosol solution, such as the product Atrovent Inhalation Aerosol (Boehringer Ingelheim). Adjustments in dosages for administration by other modes of inhaled administration are well known to those skilled in the art.

In general, an amount effective or sufficient to reduce or inhibit at least one symptom of an obstructive respiratory disease of ester compound of formula (1) or (2) is from about 1 μg to about 200 μg, e.g., from about 20 μg to about 150 μg or even from about 50 μg to about 100 μg. However, the exact dosage of the specific ester compound of formula (1) or (2) will vary depending on its potency, the mode and frequency of administration, the age and weight of the subject and the severity of the condition to be treated. The daily dosage can, for example, range from about 0.01 μg to about 10 μg per kg of body weight, administered singly or multiply in doses e.g. from about 1 μg to about 200 μg each. The compounds of formula (1) or (2) can be administered once or twice daily, preferably once daily. The amounts used are comparable to those used for glycopyrrolate (on an approximate molar basis) but cause fewer systemic side effects.

In the combined compositions, the amount of zwitterion (i)-(x) is sufficient to prolong the activity of the ester compound in treating at least one symptom of the obstructive respiratory disorder. Typically, the amounts used are in a molar ratio of ester compound:zwitterion of from about 2.5:1 to about 5:1. However, a weight:weight ratio of from about 2.5:1 to about 5:1 can be used to approximate the molar ratio. As an example, about 50-100 μg of ester can be combined with about 20 μg of zwitterion.

The dosage form for inhalation can be an aerosol. The minimum amount of an aerosol delivery is about 0.2 ml and the maximum aerosol delivery is about 5 ml. The concentration of the compounds of formula (1) or (2) and, optionally, the zwitterions (i)-(x) can vary as long as the total amount of spray delivered is within the about 0.2 to about 5 ml amount and as long as it delivers an effective amount of the compound of formula (1) or (2) as defined herein and, optionally, a sufficient amount of zwitterion as defined herein. It is well-known to those skilled in the art that if the concentration is higher, one gives a smaller dose to deliver the same effective amount.

The dosage form for inhalation can also be via intranasal spray, particularly for treatment of allergic rhinitis or infectious rhinitis. The minimum amount of an aerosol delivery is about 0.02 ml per nostril and the maximum aerosol delivery is about 0.2 ml per nostril. The concentration of the ester compounds of formula (1) or (2) and, optionally, the zwitterions (i)-(x) can vary as long as the total amount of spray delivered is within about 0.02 ml per nostril to about 0.2 ml per nostril, e.g., between about 0.05 ml per nostril and about 0.08 ml per nostril, and it delivers an effective amount of the ester compound of formula (1) or (2) as defined herein and, optionally, a sufficient amount of zwitterion (i)-(x) as defined herein.

Of course, the volume of aerosol or intranasal spray for delivering an effective amount of the ester compound of formula (1) or (2) and, optionally, a sufficient amount of the zwitterion (i)-(x) depends upon the concentration of the compound in the aerosol or intranasal spray, i.e., higher concentrations of the compound of formula (1) or (2) and, optionally, the zwitterion (i)-(x) require smaller dosage volumes to deliver a therapeutically effective and sufficient amount, respectively, and lower concentrations of the compound of formula (1) or (2) and, optionally, the zwitterion (i)-(x) require larger dosage volumes to deliver the same effective amount and sufficient amount, respectively.

Aerosols for inhalation of various pharmaceutical agents are well-known to those skilled in the art, including many aerosols for treating asthma. Aerosols can be produced with a nebulizer. Typically, the nebulizer is charged with a carrier solution and the compound of formula (1) or (2) in an amount sufficient to effectively deliver an effective amount of the ester compound of formula (1) or (2) as defined herein, optionally, together with a zwitterion (i)-(x) in an amount sufficient to prolong the activity of the ester compound. For instance, depending upon the nebulizer and its operating conditions, the nebulizer can be charged with several hundred mg of active compound in order to deliver about 1 μg to about 200 μg, preferably, from about 20 μg to about 150 μg or more preferably from about 50 μg to about 100 μg, of the compound of formula (1) or (2) and, optionally, an amount of zwitterion (i)-(x) such that the ratio of ester compound:zwitterion is from about 2.5:1 to about 5:1 by weight or molar ratio. The nebulizer can alternatively be charged with a unit dose of the compound of formula (1) or (2) and a unit dose of the zwitterion (i)-(x). As noted previously, the amount of the compound of formula (1) or (2) is about the same, on an approximate molar basis, as that used for glycopyrrolate, to obtain a substantially similar effect in treating the obstructive respiratory disease for as infrequently as once in 24 hours, but with fewer systemic side effects than glycopyrrolate.

The dosage form for inhalation can also be, and preferably is, in powder form. Powders for inhalation of various pharmaceutical agents are well-known to those skilled in the art, including many powders for treating asthma. When the dosage form is a powder, the ester compounds of formula (1) or (2) can be administered in pure form or diluted with an inert carrier. When an inert carrier is used, the ester compounds are compounded such that the total amount of powder delivered delivers an “effective amount” of the ester compound of formula (1) or (2). The actual concentration of the active ester compound can vary. If the concentration is lower, then more powder must be delivered; if the concentration is higher, less total material must be delivered to provide an effective amount of the active ester compound of formula (1) or (2). It is well-known that inhalation powder hard capsules contain a greater amount of active than the delivered dose. For example, the Novartis Seebri Breezhaler® 50 μg contains 63 μg glycopyrronium bromide equivalent to 50 μg glycopyrronium, while the delivered dose is equivalent to 44 μg glycopyrronium. Of particular interest herein are inhalation powder hard capsule unit dosage forms containing and delivering the same amounts of compound of formula (1) or (2) as the Novartis Seebri Breezhaler® does of its active ingredient, glycopyrrolate, on an approximate molar basis. Moreover, any of the foregoing pharmaceutical compositions can further comprise one or more additional active substances, particularly corticosteroids and/or betamimetics as discussed earlier.

The powder dosage form for inhalation discussed in the preceding paragraph further comprises a zwitterion (i)-(x) in an amount sufficient to prolong the activity of the ester drug, in a molar ratio or weight ratio of from about 5:1 ester:zwitterion to about 2.5:1 ester:zwitterion.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

Of particular importance when treating asthma or COPD or other serious respiratory disorders is the administration of the ester compounds or combinations of the ester compounds and the zwitterions by inhalation.

The preparations are administered by the usual methods, preferably by oral inhalation in the treatment of asthma or COPD or other serious respiratory disorders.

The dosage of the compounds of formulas (1) and (2) and, optionally, the zwitterions (i)-(x) is naturally greatly dependent on the route of administration and the complaint to be treated. When administered by oral inhalation, the compounds of formulas (1) and (2) are characterized by high efficacy even at doses in the μg range.

Embodiments herein provide combinations of a compound of formula (1) or (2) with other active agents, especially one or more antiinflammatory corticosteroids, betamimetic agents or antiallergic agents for use in the subject method. In the combination products, the active agents are present in a combined amount effective to treat the target condition, that is, to treat an obstructive disease of the respiratory tract, most especially to treat chronic obstructive pulmonary disease or asthma. In preferred embodiments, the other active agent is a betamimetic agent or antiinflammatory corticosteroid. Of particular interest are combinations of a compound of formula (1) or (2) and a corticosteroid, especially loteprednol etabonate or etiprednol dichloracetate. When loteprednol etabonate (LE) is selected as the corticosteroid, its activity can be enhanced by combination with cortienic acid or Δ¹-cortienic acid or a methyl or ethyl ester of cortienic acid or a methyl or ethyl ester of Δ¹-cortienic acid, in a mole ratio of LE:enhancer of from about 5:1 to about 0.5:1. A molar ratio of about 1:1, which can be approximated by a 1:1 ratio by weight, is particularly convenient.

Particular embodiments herein provide combinations of a compound of formula (1) or (2) and zwitterion (i)-(x) with other active agents, especially one or more antiinflammatory corticosteroids, betamimetic agents or antiallergic agents. In these further combination products, the active agents (not including the zwitterion) are present in a combined amount effective to treat the target condition, that is, to treat an obstructive disease of the respiratory tract, most especially to treat chronic obstructive pulmonary disease or asthma. In preferred embodiments, the further active agent is a betamimetic agent or antiinflammatory corticosteroid. Of particular interest, are combinations of an ester compound of formula (1) or (2), a zwitterion (i)-(x), and a corticosteroid, especially loteprednol etabonate or etiprednol dichloracetate. When loteprednol etabonate (LE) is selected as the corticosteroid, its activity can be enhanced by combination with cortienic acid or Δ¹-cortienic acid or a methyl or ethyl ester of cortienic acid or a methyl or ethyl ester of Δ¹-cortienic acid, in a mole ratio of LE:enhancer of from about 5:1 to about 0.5:1. A molar ratio of about 1:1, which can be approximated by a 1:1 ratio by weight, is particularly convenient.

One embodiment provides a combination of the compounds of formulas (1) and (2) with zwitterions (i)-(x), with one or more further active agents, and compositions comprising a compound of formula (1) or (2) and a zwitterion (i)-(x), with or without one or more other active agents and carrier, as described hereinabove, are thus useful in a method for treating an obstructive disease of the respiratory tract in a subject in need of such treatment, comprising administering to said subject, once or twice daily, an amount of said compound, combination or composition effective to inhibit or diminish at least one symptom of said disease, the amount of zwitterion being as defined earlier herein. Use of compounds of formula (1) or (2) together with zwitterions (i)-(x), in the preparation of a medicament for treating such a condition is likewise provided herein, as is a combination composition for use in such treatment.

Alternatively, provided herein is a method for treating an obstructive disease of the respiratory tract in a subject in need of such treatment comprising separately:

(a) Administering an ester compound of formula (1) or (2) as defined herein, or a composition comprising an ester compound of formula (1) or (2) and a non-toxic pharmaceutically acceptable, preferably anhydrous, carrier or excipient therefor, wherein the amount of ester compound of formula (1) or (2) is an amount effective to reduce or inhibit at least one symptom of said obstructive disease, once or twice daily, by oral inhalation;

(b) Administering a zwitterion (i)-(x) as defined herein, or a composition comprising a zwitterion (i)-(x) and a non-toxic pharmaceutically acceptable carrier or excipient therefor, wherein the amount of zwitterion (i)-(x) is sufficient to prolong the activity of the ester compound in reducing or inhibiting at least one symptom of said obstructive disease, once or twice daily, by oral inhalation.

The compositions administered according to the method of part (a) of the preceding paragraph, which are preferably inhalable powders, are prepared in the same way as the composition described hereinabove, save for the absence of the zwitterion (i)-(x). The composition administered according to part (b) of the preceeding paragraph, which likewise are preferably inhalable powders, are prepared in a similar way to the compositions described hereinabove; however, because of the absence of the compounds of formula (1) or (2), the compositions used in part (b) do not need to be prepared or maintained in anhydrous form (because the zwitterions themselves are water-soluble). Moreover, this alternative method of accomplishing the desired treatment allows for a number of possibilities not provided by co-administering the combination of ester compound and the zwitterion in a single dosage form:

1. The separate administration of the zwitterion and of the ester compound can take place at different times of day, if desired, for example, one in the morning and the other in the evening, or one following the other, separately by a time period of from several minutes to eight or more hours. In this way, the subject's system could be primed by first administering a dose (or multiple doses) of the zwitterion to build its concentration;

2. The frequency of the separate administration of each of the two actives, zwitterion and ester compound, can differ, if desired. For example, after the concentration of the longer-acting zwitterion has been built up sufficiently, the ester compound could be administered more frequently than the zwitterion;

3. The unit dosage forms can be manufactured in accord with the water solubility properties of each of the two required actives.

Powders for inhalation are preferred for use in treatment of COPD, asthma and other serious obstructive diseases of the respiratory tract as described herein. The preparation of these powders can make use of methods previously described for the preparation of inhalable powders of glycopyrrolate or glycopyrronium bromide for treatment of respiratory diseases.

See, in particular, Bannister et al. WO 2001/076575 and the corresponding Bannister et al. U.S. Pat. No. 7,368,104 B2, which is said to provide a pharmaceutical composition for pulmonary delivery which comprises glycopyrrolate in a controlled release formulation wherein, on administration, the glycopyrrolate exerts its pharmacological effect over a period greater than 12 hours. In a particular embodiment a dry powder composition comprising microparticles of glycopyrrolate is said to exert its pharmacological effect for a period greater than 20 hours, preferably using large carrier particles which are lactose particles and a hydrophobic material such as magnesium stearate.

See also, Morton et al. WO 2005/025536, which teaches a process for producing dry powder compositions that purportedly have physical and chemical properties that provide an enhanced fine particle fraction (FPF) and fine particle dose (FPD), which is said to provide greater dosing efficiency. Morton et al.'s method comprises co-jet milling active particles of drug (such as glycopyrrolate) in the presence of an additive material. The active can be first jet-milled alone to a small particle size, then blended with the additive and then co-jet milled with the additive at a lower grinding pressure to coat the small active drug particles with the additive. Morton et al. teach that the additive can be an amino acid, a phospholipid, a metal stearate such as magnesium stearate or a surfactant.

In addition, Morton et al. WO 2005/105043 and its corresponding Morton et al. United States Patent Publication No. 2008/0063719 A1, describe the preparation of dry powder pharmaceutical compositions comprising glycopyrrolate, for example glycopyrronium bromide, their purported improved stability over time and methods for producing them.

Moreover, Haeberlin et al. WO 2008/000482 and its corresponding Haeberlin et al. United States Patent Application Publication No. 2012/0065174 A1 and Haeberlin et al. European Patent Specification EP 2037879 B1, provide a process for preparing dry powder formulations of a glycopyrronium salt for inhalation that is taught to have good stability. The process involves: (1) admixing a glycopyrronium salt together with an anti-adherent agent to give a homogeneous blend; (b) micronizing the blend; and (c) admixing carrier particles to form a dry powder formulation, wherein the carrier particles are mixed with the blend in a ratio of 2000:1 to 5:1 by mass. The anti-adherent agent is taught to be a metal stearate, a crystalline sugar or a mixture thereof. The metal stearate can be magnesium stearate or calcium stearate. The carrier particles can be crystalline sugars.

All of the Bannister et al., Morton et al. and Haeberlin et al. patent documents referred to above are incorporated by reference herein in their entireties.

Adaptation of these methods and compositions to the combination compounds of formulas (1) and (2) and zwitterions (i)-(x) herein is shown in the EXAMPLES which follow.

The following EXAMPLES further illustrate compositions for use in the methods described herein. These are illustrative and are not to be considered limiting in any way whatsoever, as many modifications in materials and methods will be apparent to those skilled in the art.

### ESTER FORMULATION EXAMPLES

### Example 1

| Metering aerosol | % by weight |
|---|---|
| Ester compound of formula (1), e.g. | 0.005 |
| Compound 5, 8, 27, 31, or 35 | |
| Sorbitan trioleate | 0.1 |
| Monofluorotrichloromethane and | 2:3 ad 100 |
| difluorodichloromethane | |

The suspension is transferred into a conventional aerosol container with a metering valve. Preferably, 50 μl of suspension are delivered per spray. The active substance may also be metered in higher doses if desired (e.g. 0.01% by weight).

### Example 2

| Solution (in mg/100 ml) | |
|---|---|
| Ester compound of formula (1), e.g. | 333.3 mg |
| Compound 5, 8, 27, 31, or 35 | |
| Formoterol fumarate | 333.3 mg |
| Benzalkonium chloride | 10.0 mg |
| EDTA | 50.0 mg |
| HCl(In) | ad pH 3.4 |

This solution may be prepared in the usual manner.

### Example 3

| Powder for inhalation | μg per dosage unit |
|---|---|
| Ester compound of formula (1), e.g. | 60 μg |
| Compound 5, 8, 27, 31, or 35 | |
| (micronized) | |
| Formoterol fumarate | 60 μg |
| Magnesium stearate | 0.15% to 0.25% by weight |
| Lactose | ad 25 mg |

The powder for inhalation is produced according to the method of Morton et al. US2008/0063719 by mixing the individual ingredients together, with the micronized active added last. Morton et al US2008/0063719 is incorporated by reference herein in its entirety and relied upon.

### Example 4

| Powder for inhalation | μg per dosage unit |
|---|---|
| Ester compound of formula (1), e.g. | 60 μg |
| Compound 5, 8, 27, 31, or 35 | |
| (micronized) | |
| Magnesium stearate | 0.15% to 0.25% by weight |
| Lactose | ad 5 mg |

The powder for inhalation is produced as in Example 3 by the method of Morton et al., by mixing the individual ingredients together, adding the micronized active last.

### Further Formulations Obtained Analogously to Methods Known in the Art

A: Inhalable powders are prepared according to the method of Haeberlin et. al., EP2037879 B1 and US2012/0065174 A1 by mixing the formula (1) salt with magnesium stearate to give a homogeneous blend, micronizing the blend and admixing the remaining ingredients therein. Alternatively, when a second active ingredient is present, it too can be micronized with the compound of formula (1) and the magnesium stearate in the first step. US2012/0065174 A1 and EP 2037879 B1 are incorporated by reference herein in their entireties and relied upon.

### Example 5

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Budesonide | 120 |
| Lactose (anhydrous) | q.s. to 5000 |

### Example 6

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Fluticasone propionate | 100 |
| Lactose (anhydrous) | q.s. to 5000 |

### Example 7

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Ciclesonide | 250 |
| Lactose (anhydrous) | q.s. to 5000 |

### Example 8

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Budesonide | 125 |
| Lactose (anhydrous) | q.s. to 5000 |

### Example 9

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Fluticasone propionate | 200 |
| Lactose (anhydrous) | q.s. to 5000 |

### Example 10

| Ingredients | μg per capsule |
|---|---|
| Ester compound of formula (1), e.g. | 75 |
| Compound 5, 8, 27, 31, or 35 | |
| Magnesium stearate | 0.15% to 0.25% by weight |
| Ciclesonide | 250 |
| Lactose (anhydrous) | q.s. to 5000 |

### Example 11

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Etiprednol dichloracetate | 250 |
| Lactose (anhydrous) | q.s. to 5000 |

### Example 12

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Loteprednol etabonate | 125 |
| Lactose (anhydrous) | q.s. to 5000 |

### Example 13

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 200 |
| Lactose (anhydrous) | q.s. to 5000 |

### Example 14

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Etiprednol dichloracetate | 200 |
| Lactose (anhydrous) | q.s. to 5000 |

### Example 15

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 125 |
| Lactose (anhydrous) | q.s. to 5000 |

### Example 16

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Etiprednol dichloracetate | 125 |
| Lactose (anhydrous) | q.s. to 5000 |

### Example 17

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 200 |
| Δ¹-Cortienic acid methyl ester | 200 |
| Lactose (anhydrous) | q.s. to 5000 |

### Example 18

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 200 |
| Δ¹-Cortienic acid | 200 |
| Lactose (anhydrous) | q.s. to 5000 |

### Example 19

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Loteprednol etabonate | 125 |
| Δ¹-Cortienic acid or | 125 |
| Δ¹-Cortienic acid methyl ester | |
| Lactose (anhydrous) | q.s. to 5000 |

### Example 20

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Loteprednol etabonate | 125 |
| Δ¹-Cortienic acid or | 125 |
| Δ¹-Cortienic acid methyl ester | |
| Lactose (anhydrous) | q.s. to 5000 |

B. Propellant-Containing Aerosols for Inhalation (wherein TG 134a is 1,1,1,2-tetrafluoroethane and TG 227 is 1,1,1,2,3,3,3-heptafluoropropane)

### Example 21: Suspension Aerosol

| Ingredients | % by weight |
|---|---|
| Ester compound of formula (1), e.g. | 0.015 |
| Compound 5, 8, 27, 31, or 35 | |
| Budesonide | 0.4 |
| Soya lecithin | 0.2 |
| TG 134a:TG 227 (2:3) | to 100 |

### Example 22: Suspension Aerosol

| Ingredients | % by weight |
|---|---|
| Ester compound of formula (1), e.g. | 0.015 |
| Compound 5, 8, 27, 31, or 35 | |
| Fluticasone propionate | 0.3 |
| Isopropyl myristate | 0.1 |
| TG 227 | to 100 |

### Example 23: Suspension Aerosol

| Ingredients | % by weight |
|---|---|
| Ester compound of formula (1), e.g. | 0.015 |
| Compound 5, 8, 27, 31, or 35 | |
| Ciclesonide | 0.4 |
| Isopropyl myristate | 0.1 |
| TG 134a:TG 227 (2:3) | to 100 |

### Example 24: Suspension Aerosol

| Ingredients | % by weight |
|---|---|
| Ester compound of formula (1), e.g. | 0.010 |
| Compound 5, 8, 27, 31, or 35 | |
| Ciclesonide | 0.4 |
| Isopropyl myristate | 0.1 |
| TG 134a:TG 227 (2:3) | to 100 |

### Example 25: Aerosol

| Ingredients | % by weight |
|---|---|
| Ester compound of formula (1), e.g. | 0.015 |
| Compound 5, 8, 27, 31, or 35 | |
| Fluticasone propionate | 0.2 |
| Isopropyl myristate | 0.1 |
| TG 134a:TG 227 (2:3) | to 100 |

### Example 26: Aerosol

| Ingredients | % by weight |
|---|---|
| Ester compound of formula (1), e.g. | 0.020 |
| Compound 5, 8, 27, 31, or 35 | |
| Ciclesonide | 0.4 |
| Isopropyl myristate | 0.1 |
| TG 134a:TG 227 (2:3) | to 100 |

### Example 27

| Ingredients | % by weight |
|---|---|
| Ester compound of formula (1), e.g. | 0.010 |
| Compound 5, 8, 27, 31, or 35 | |
| Loteprednol etabonate | 0.4 |
| Soya lecithin | 0.2 |
| TG 134a:TG 227 (2:3) | to 100 |

### Example 28

| Ingredients | % by weight |
|---|---|
| Ester compound of formula (1), e.g. | 0.015 |
| Compound 5, 8, 27, 31, or 35 | |
| Loteprednol etabonate | 0.3 |
| Isopropyl myristate | 0.1 |
| TG 227 | to 100 |

### Example 29

| Ingredients | % by weight |
|---|---|
| Ester compound of formula (1), e.g. | 0.010 |
| Compound 5, 8, 27, 31, or 35 | |
| Etiprednol dichloracetate | 0.4 |
| Isopropyl myristate | 0.1 |
| TG 227 | to 100 |

### Example 30

| Ingredients | % by weight |
|---|---|
| Ester compound of formula (1), e.g. | 0.010 |
| Compound 5, 8, 27, 31, or 35 | |
| Loteprednol etabonate | 0.4 |
| Isopropyl myristate | 0.1 |
| TG 134a:TG 227 (2:3) | to 100 |

### Example 31

| Ingredients | % by weight |
|---|---|
| Ester compound of formula (1), e.g. | 0.015 |
| Compound 5, 8, 27, 31, or 35 | |
| Loteprednol etabonate | 0.4 |
| Isopropyl myristate | 0.1 |
| TG 134a:TG 227 (2:3) | to 100 |

### Example 32

| Ingredients | % by weight |
|---|---|
| Ester compound of formula (1), e.g. | 0.015 |
| Compound 5, 8, 27, 31, or 35 | |
| Loteprednol etabonate | 0.4 |
| Δ¹-Cortienic acid or | 0.4 |
| Δ¹-Cortienic acid methyl ester | |
| Soya lecithin | 0.2 |
| TG 134a:TG 227 (2:3) | to 100 |

### Example 33

| Ingredients | % by weight |
|---|---|
| Ester compound of formula (1), e.g. | 0.010 |
| Compound 5, 8, 27, 31, or 35 | |
| Loteprednol etabonate | 0.3 |
| Δ¹-Cortienic acid or | 0.3 |
| Δ¹-Cortienic acid methyl ester | |
| Isopropyl myristate | 0.1 |
| TG 227 | to 100 |

### Example 34

| Ingredients | % by weight |
|---|---|
| Ester compound of formula (1), e.g. | 0.015 |
| Compound 5, 8, 27, 31, or 35 | |
| Loteprednol etabonate | 0.4 |
| Δ¹-Cortienic acid or | 0.4 |
| Δ¹-Cortienic acid methyl ester | |
| Isopropyl myristate | 0.1 |
| TG 227 | to 100 |

### Example 35

| Ingredients | % by weight |
|---|---|
| Ester compound of formula (1), e.g. | 0.015 |
| Compound 5, 8, 27, 31, or 35 | |
| Loteprednol etabonate | 0.4 |
| Δ¹-Cortienic acid or | 0.4 |
| Δ¹-Cortienic acid methyl ester | |
| Isopropyl myristate | 0.1 |
| TG 134a:TG 227 (2:3) | to 100 |

### Example 36

| Ingredients | % by weight |
|---|---|
| Ester compound of formula (1), e.g. | 0.010 |
| Compound 5, 8, 27, 31, or 35 | |
| Loteprednol etabonate | 0.4 |
| Δ¹-Cortienic acid or | 0.4 |
| Δ¹-Cortienic acid methyl ester | |
| Isopropyl myristate | 0.1 |
| TG 134a:TG 227 (2:3) | to 100 |

Yet other compositions can be conveniently formulated using known techniques.

### Investigation of the Anticholinergic Action of Compounds in Acetylcholine Induced Bronchoconstriction in Anesthetized Guinea Pigqs

### Experimental Procedure

Male Hartley guinea pigs (320±120 g) (Charles River) are housed under standard conditions. Guinea pigs are anesthetized with urethane (2 g/kg, intraperitoneally), the trachea are cannulated and the animal is respired using a small animal respiratory pump (Harvard Apparatus LTD, Kent UK). Respiratory back pressure is measured and recorded using a rodent lung function recording system (MUMED, London UK). For drug administration the right jugular vein is cannulated. Following the surgical procedure, guinea pigs are allowed to stabilize for 20 minutes. Ten minutes before acetylcholine administration, the animals are disconnected from the ventilator and either the vehicle (10 mg lactose) or different amounts of the drug (suspended in the same amount of vehicle) are administered intratracheally. The trachea is reconnected to the ventilator and changes in pulmonary mechanics are followed. Acetylcholine (10 μg/kg) is administered intravenously in every 10 minutes six times.

### Results

Compounds of formula (1) and (2) and glycopyrrolate will exhibit a protective effect on the acetylcholine-induced bronchoconstriction provoked in this test.

This test is a model for asthma, chronic obstructive pulmonary disorder and other obstructive respiratory tract disorders in which the effectiveness of the compounds of formulas (1) and (2) can be evaluated.

### Test for Bronchodilatory Effect of Inhaled Test Compounds in Balb/c Mice

Female BALB/c mice, weight range 19-22 g, are obtained, for example from Charles River Laboratories (Kingston, N.C.). They receive food and water ad libitum.

Compounds for aerosol administration are prepared in sterile Dulbecco's Phosphate Buffered Saline. Mice are placed on a carousel-style, nose only, exposure chamber and allowed to inhale aerosols for five minutes, using an ICN SPAG-2 nebulizer. This nebulizer generates a mean aerosol particle size of 1.3 microns at a rate of approximately 0.25 ml/minute.

Ten minutes and 36 hours later, the mice are moved to whole body plethysmograph chambers. Bronchoconstriction is induced in the mice by administration of an 80 mg/ml methacholine (MC) aerosol in the plethysmograph chambers for 5 minutes. The mice are allowed to inhale an aerosol containing 80 mg/ml methacholine following inhalation treatment with DPBS vehicle (Dulbecco's Phosphate Buffered Saline), or 80 mg/ml methacholine following inhalation treatment with test compound. The average enhanced pause (Penh, lung resistance), corresponding to airflow resistance, is determined and statistically analyzed using Kruskal-Wallis one way ANOVA. In order to determine the baseline, saline aerosol (without methacholine) is also separately administered to the mice.

This procedure is a model for inhalation treatment of asthma, chronic obstructive pulmonary disorder and other obstructive respiratory tract disorders in which the Effectiveness of the Compounds of Formulas (1) or (2) can be Tested.

### Ester-Zwitterion Combination Examples

### Example 37

| Metering aerosol | % by weight |
|---|---|
| Ester compound of formula (1), e.g. | 0.005 |
| Compound 5, 8, 27, 31, or 35 | |
| Zwitterion, e.g. (vii) [BOD-08] | 0.001 to 0.0025 |
| Sorbitan trioleate | 0.1 |
| Monofluorotrichloromethane and | ad 100 |
| difluorodichloromethane (2:3) | |

The suspension is transferred into a conventional aerosol container with a metering valve. Preferably, 50 Jul of suspension are delivered per spray. The active substances may also be metered in higher doses if desired (e.g. 0.01% by weight of ester compound and 0.005% by weight of zwitterion).

### Example 38

| Solution (in mg/100 ml) | |
|---|---|
| Ester compound of formula (1), e.g. | 333.3 mg |
| Compound 5, 8, 27, 31, or 35 | |
| Zwitterion, e.g. (vii) [BOD-08] | 33.3 mg-66.7 mg |
| Formoterol fumarate | 333.3 mg |
| Benzalkonium chloride | 10.0 mg |
| EDTA | 50.0 mg |
| HCl(In) | ad pH 3.4 |

This solution may be prepared in the usual manner.

### Example 39

| Powder for inhalation | μg per dosage unit | |
|---|---|---|
| Ester compound of formula (1), e.g. | 60 | μg |
| Compound 5, 8, 27, 31, or 35 | | |
| (micronized) | | |
| Zwitterion, e.g. (vii) [BOD-08] | 12-24 | μg |
| Formoterol fumarate | 60 | μg |
| Magnesium stearate | 0.15% to 0.25% | by weight |
| Lactose | ad 25 | mg |

The powder for inhalation is produced according to the method of Morton et al. US2008/0063719 by mixing the individual ingredients together, with the micronized actives added last. Morton et al US2008/0063719 is incorporated by reference herein in its entirety and relied upon.

### Example 40

| Powder for inhalation | μg per dosage unit | |
|---|---|---|
| Ester compound of formula (1), e.g. | 60 | μg |
| Compound 5, 8, 27, 31, or 35 | | |
| (micronized) | | |
| Zwitterion, e.g. (vii) [BOD-08] | 12-24 | μg |
| Magnesium stearate | 0.15% to 0.25% | by weight |
| Lactose | ad 5 | mg |

The powder for inhalation is produced as in Example 39 by the method of Morton et al., by mixing the individual ingredients together, adding the micronized actives last.

### Further Formulations Obtained Analogously to Methods Known in the Art

Inhalable powders prepared according to the method of Haeberlin et. al., EP2037879 B1 and US2012/0065174 A1 by mixing the formula (1) salt and the zwitterion with magnesium stearate to give a homogeneous blend, micronizing the blend and admixing the remaining ingredients therein. Alternatively, when a further active ingredient is present, it too can be micronized with the compound of formula (1), the zwitterion and the magnesium stearate in the first step. US2012/0065174 A1 and EP 2037879 B1 are incorporated herein in their entireties and relied upon.

### Example 41

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Budesonide | 120 |
| Lactose (anhydrous) | q.s. to 5000 |

### Example 42

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Fluticasone propionate | 100 |
| Lactose (anhydrous) | q.s. to 5000 |

### Example 43

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Ciclesonide | 250 |
| Lactose (anhydrous) | q.s. to 5000 |

### Example 44

| Ingredients | μg per capsule |
|---|---|
| Ester compound of formula (1), e.g. | 50 |
| Compound 5, 8, 27, 31, or 35 | |
| Zwitterion, e.g. (vii) [BOD-08] | 10-20 |
| Magnesium stearate | 0.15% to 0.25% by weight |
| Budesonide | 125 |
| Lactose (anhydrous) | q.s. to 5000 |

### Example 45

| Ingredients | μg per capsule |
|---|---|
| Ester compound of formula (1), e.g. | 50 |
| Compound 5, 8, 27, 31, or 35 | |
| Zwitterion, e.g. (vii) [BOD-08] | 10-20 |
| Magnesium stearate | 0.15% to 0.25% by weight |
| Fluticasone propionate | 200 |
| Lactose (anhydrous) | q.s. to 5000 |

### Example 46

| Ingredients | μg per capsule |
|---|---|
| Ester compound of formula (1), e.g. | 75 |
| Compound 5, 8, 27, 31, or 35 | |
| Zwitterion, e.g. (vii) [BOD-08] | 15-30 |
| Magnesium stearate | 0.15% to 0.25% by weight |
| Ciclesonide | 250 |
| Lactose (anhydrous) | q.s. to 5000 |

### Example 47

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Etiprednol dichloracetate | 250 |
| Lactose (anhydrous) | q.s. to 5000 |

### Example 48

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Loteprednol etabonate | 125 |
| Lactose (anhydrous) | q.s. to 5000 |

### Example 49

| Ingredients | μg per capsule |
|---|---|
| Ester compound of formula (1), e.g. | 100 |
| Compound 5, 8, 27, 31, or 35 | |
| Zwitterion, e.g. (vii) [BOD-08] | 20-40 |
| Magnesium stearate | 0.15% to 0.25% by weight |
| Loteprednol etabonate | 200 |
| Lactose (anhydrous) | q.s. to 5000 |

### Example 50

| Ingredients | μg per capsule |
|---|---|
| Ester compound of formula (1), e.g. | 100 |
| Compound 5, 8, 27, 31, or 35 | |
| Zwitterion, e.g. (vii) [BOD-08] | 20-40 |
| Magnesium stearate | 0.15% to 0.25% by weight |
| Etiprednol dichloracetate | 200 |
| Lactose (anhydrous) | q.s. to 5000 |

### Example 51

| Ingredients | μg per capsule |
|---|---|
| Ester compound of formula (1), e.g. | 100 |
| Compound 5, 8, 27, 31, or 35 | |
| Zwitterion, e.g. (vii) [BOD-08] | 20-40 |
| Magnesium stearate | 0.15% to 0.25% by weight |
| Loteprednol etabonate | 125 |
| Lactose (anhydrous) | q.s. to 5000 |

### Example 52

| Ingredients | μg per capsule |
|---|---|
| Ester compound of formula (1), e.g. | 50 |
| Compound 5, 8, 27, 31, or 35 | |
| Zwitterion, e.g. (vii) [BOD-08] | 10-20 |
| Magnesium stearate | 0.15% to 0.25% by weight |
| Etiprednol dichloracetate | 125 |
| Lactose (anhydrous) | q.s. to 5000 |

### Example 53

| Ingredients | μg per capsule |
|---|---|
| Ester compound of formula (1), e.g. | 100 |
| Compound 5, 8, 27, 31, or 35 | |
| Zwitterion, e.g. (vii) [BOD-08] | 20-40 |
| Magnesium stearate | 0.15% to 0.25% by weight |
| Δ¹-Cortienic acid methyl ester | 200 |
| Lactose (anhydrous) | q.s. to 5000 |

### Example 54

| Ingredients | μg per capsule |
|---|---|
| Ester compound of formula (1), e.g. | 100 |
| Compound 5, 8, 27, 31, or 35 | |
| Zwitterion, e.g. (vii) [BOD-08] | 20-40 |
| Magnesium stearate | 0.15% to 0.25% by weight |
| Loteprednol etabonate | 200 |
| Δ¹-Cortienic acid | 200 |
| Lactose (anhydrous) | q.s. to 5000 |

### Example 55

| Ingredients | μg per capsule |
|---|---|
| Ester compound of formula (1), e.g. | 60 |
| Compound 5, 8, 27, 31, or 35 | |
| Zwitterion, e.g. (vii) [BOD-08] | 12-24 |
| Magnesium stearate | 0.15% to 0.25% by weight |
| Loteprednol etabonate | 125 |
| Δ¹-Cortienic acid or | 200 |
| Δ¹-Cortienic acid methyl ester | |
| Lactose (anhydrous) | q.s. to 5000 |

### Example 56

| Ingredients | μg per capsule |
|---|---|
| Ester compound of formula (1), e.g. | 50 |
| Compound 5, 8, 27, 31, or 35 | |
| Zwitterion, e.g. (vii) [BOD-08] | 10-20 |
| Magnesium stearate | 0.15% to 0.25% by weight |
| Loteprednol etabonate | 125 |
| Δ¹-Cortienic acid or | 125 |
| Δ¹-Cortienic acid methyl ester | |
| Lactose (anhydrous) | q.s. to 5000 |

B. Propellant-Containing Aerosols for Inhalation (wherein TG 134a is 1,1,1,2-tetrafluoroethane and TG 227 is 1,1,1,2,3,3,3-heptafluoropropane)

### Example 57: Suspension Aerosol

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Budesonide | 0.4 |
| Soya lecithin | 0.2 |
| TG 134a:TG 227 (2:3) | to 100 |

### Example 58: Suspension Aerosol

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Fluticasone propionate | 0.3 |
| Isopropyl myristate | 0.1 |
| TG 227 | to 100 |

### Example 59: Suspension Aerosol

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Ciclesonide | 0.4 |
| Isopropyl myristate | 0.1 |
| TG 134a:TG 227 (2:3) | to 100 |

### Example 60: Suspension Aerosol

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Ciclesonide | 0.4 |
| Isopropyl myristate | 0.1 |
| TG 134a:TG 227 (2:3) | to 100 |

### Example 61: Aerosol

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Fluticasone propionate | 0.2 |
| Isopropyl myristate | 0.1 |
| TG 134a:TG 227 (2:3) | to 100 |

### Example 62: Aerosol

| Ingredients | % by weight |
|---|---|
| Ester compound of formula (1), e.g. | 0.020 |
| Compound 5, 8, 27, 31, or 35 | |
| Zwitterion, e.g. (vii) [BOD-08] | 0.004-0.008 |
| Ciclesonide | 0.4 |
| Isopropyl myristate | 0.1 |
| TG 134a:TG 227 (2:3) | to 100 |

### Example 63

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Loteprednol etabonate | 0.4 |
| Soya lecithin | 0.2 |
| TG 134a:TG 227 (2:3) | to 100 |

### Example 64

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Loteprednol etabonate | 0.3 |
| Isopropyl myristate | 0.1 |
| TG 227 | to 100 |

### Example 65

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Etiprednol dichloracetate | 0.4 |
| Isopropyl myristate | 0.1 |
| TG 227 | to 100 |

### Example 66

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Loteprednol etabonate | 0.4 |
| Isopropyl myristate | 0.1 |
| TG 134a:TG 227 (2:3) | to 100 |

### Example 67

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Loteprednol etabonate | 0.4 |
| Isopropyl myristate | 0.1 |
| TG 134a:TG 227 (2:3) | to 100 |

### Example 68

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Loteprednol etabonate | 0.4 |
| Δ¹-Cortienic acid or | 0.2 |
| Δ¹-Cortienic acid methyl ester | |
| Soya lecithin | 0.2 |
| TG 134a:TG 227 (2:3) | to 100 |

### Example 69

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Loteprednol etabonate | 0.3 |
| Δ¹-Cortienic acid or | 0.3 |
| Δ¹-Cortienic acid methyl ester | |
| Isopropyl myristate | 0.1 |
| TG 227 | to 100 |

### Example 70

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Loteprednol etabonate | 0.4 |
| Δ¹-Cortienic acid or | 0.4 |
| Δ¹-Cortienic acid methyl ester | |
| Isopropyl myristate | 0.1 |
| TG 227 | to 100 |

### Example 71

| Ingredients | % by weight |
|---|---|
| Ester compound of formula (1), e.g. | 0.015 |
| Compound 5, 8, 27, 31, or 35 | |
| Zwitterion, e.g. (vii) [BOD-08] | 0.003-0.006 |
| Loteprednol etabonate | 0.4 |
| Δ¹-Cortienic acid or | 0.4 |
| Δ¹-Cortienic acid methyl ester | |
| Isopropyl myristate | 0.1 |
| TG 134a:TG 227 (2:3) | to 100 |

### Example 72

| Ingredients | % by weight |
|---|---|
| Ester compound of formula (1), e.g. | 0.010 |
| Compound 5, 8, 27, 31, or 35 | |
| Zwitterion, e.g. (vii) [BOD-08] | 0.002-0.004 |
| Loteprednol etabonate | 0.4 |
| Δ¹-Cortienic acid or | 0.4 |
| Δ¹-Cortienic acid methyl ester | |
| Isopropyl myristate | 0.1 |
| TG 134a:TG 227 (2:3) | to 100 |

### Example 73

Part (a) Composition

| Powder for Inhalation | μg per dosage unit | |
|---|---|---|
| Ester compound of formula (1), e.g. | 60 | μg |
| Compound 5, 8, 27, 31, or 35 | | |
| (Micronized) | | |
| Magnesium stearate | 0.15% to 0.25% | by weight |
| Lactose | ad 5 | mg |

Part (b) composition

| Powder for Inhalation | μg per dosage unit | |
|---|---|---|
| Zwitterion, e.g. (vii) [BOD-08] | 12-24 | μg |
| (Micronized) | | |
| Magnesium stearate | 0.15% to 0.25% | by weight |
| Lactose | ad 5 | mg |

Each powder is produced as in EXAMPLE 39 by the method of Morton et al., by mixing the individual ingredients together, adding the micronized actives last. These inhalable powder compositions are designed for the alternative method of treatment, in which the compositions comprising the ester compounds are administered separately from the compositions comprising the zwitterions. Thus, they can be administered with different frequencies or at different time points.

### Example 74

Part (a) Composition

| Ingredients | μg per capsule |
|---|---|
| Ester compound of formula (1), | 60 |
| e.g. Compound 5, 8, 27, 31, or 35 | |
| Magnesium stearate | 0.15% to 0.25% by weight |
| Etiprednol dichloracetate | 250 |
| Lactose (anhydrous) | q.s. to 5000 |

Part (b) Composition

| Ingredients | μg per capsule | |
|---|---|---|
| Zwitterion, e.g. (vii) [BOD-08] | 12-24 | μg |
| Magnesium stearate | 0.15% to 0.25% | by weight |

| | |
|---|---|
| Lactose (anhydrous) | q.s. to 5000 |

The method of Haeberlin et al. modified as described in [0108] above is followed, except the ester compound and the zwitterion are placed in separate compositions. Each active is mixed with magnesium stearate to give a homogenous blend, the blend is micronized and any remaining ingredients admixed therein. Alternatively, when yet another active ingredient is included, it too can be micronized with the ester compound. The inhalable powder compositions are for use as in EXAMPLE 73.

### Example 75

Part (a) Composition

| Ingredients | μg per capsule |
|---|---|
| Ester compound of formula (1), | 60 |
| e.g. Compound 5, 8, 27, 31, or 35 | |
| Magnesium stearate | 0.15% to 0.25% by weight |
| Loteprednol etabonate | 125 |
| Lactose (anhydrous) | q.s. to 5000 |

Part (b) Composition

| Ingredients | μg per capsule |
|---|---|
| Zwitterion, e.g. (vii) [BOD-08] | 12-24 |
| Magnesium stearate | 0.15% to 0.25% by weight |
| Lactose (anhydrous) | q.s. to 5000 |

These inhalable powder compositions are formulated and administered as described in EXAMPLE 74.

### Example 76

Part (a) Composition

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 200 |
| Lactose (anhydrous) | q.s. to 5000 |

Part (b) Composition

| Ingredients | μg per capsule |
|---|---|
| Zwitterion, e.g. (vii) [BOD-08] | 20-40 |
| Magnesium stearate | 0.15% to 0.25% by weight |
| Lactose (anhydrous) | q.s. to 5000 |

These inhalable powder compositions are formulated and administered as described in EXAMPLE 74.

### Example 77

Part (a) Composition

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Etiprednol dichloracetate | 200 |
| Lactose (anhydrous) | q.s. to 5000 |

Part (b) Composition

| Ingredients | μg per capsule |
|---|---|
| Zwitterion, e.g. (vii) [BOD-08] | 20-40 |
| Magnesium stearate | 0.15% to 0.25% by weight |
| Lactose (anhydrous) | q.s. to 5000 |

These inhalable powder compositions are formulated and administered as described in EXAMPLE 74.

### Example 78

Part (a) Composition

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 125 |
| Lactose (anhydrous) | q.s. to 5000 |

Part (b) Composition

| Ingredients | μg per capsule |
|---|---|
| Zwitterion, e.g. (vii) [BOD-08] | 20-40 |
| Magnesium stearate | 0.15% to 0.25% by weight |
| Lactose (anhydrous) | q.s. to 5000 |

These inhalable powder compositions are formulated and administered as described in EXAMPLE 74.

### Example 79

Part (a) Composition

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Etiprednol dichloracetate | 125 |
| Lactose (anhydrous) | q.s. to 5000 |

Part (b) Composition

| Ingredients | μg per capsule |
|---|---|
| Zwitterion, e.g. (vii) [BOD-08] | 10-20 |
| Magnesium stearate | 0.15% to 0.25% by weight |
| Lactose (anhydrous) | q.s. to 5000 |

These inhalable powder compositions are formulated and administered as described in EXAMPLE 74.

### Example 80

Part (a) Composition

| Ingredients | μg per capsule |
|---|---|
| Ester compound of formula (1), e.g. | 100 |
| Compound 5, 8, 27, 31, or 35 | |
| Magnesium stearate | 0.15% to 0.25% by weight |
| Loteprednol etabonate | 200 |
| Δ¹-Cortienic acid or | 200 |
| Δ¹-Cortienic acid methyl ester | |
| Lactose (anhydrous) | q.s. to 5000 |

Part (b) Composition

| Ingredients | μg per capsule |
|---|---|
| Zwitterion, e.g. (vii) [BOD-08] | 20-40 |
| Magnesium stearate | 0.15% to 0.25% by weight |
| Lactose (anhydrous) | q.s. to 5000 |

These inhalable powder compositions are formulated and administered as described in EXAMPLE 74.

### Example 81

Part (a) Composition

| Ingredients | μg per capsule |
|---|---|
| Ester compound of formula (1), e.g. | 60 |
| Compound 5, 8, 27, 31, or 35 | |
| Magnesium stearate | 0.15% to 0.25% by weight |
| Loteprednol etabonate | 125 |
| Δ¹-Cortienic acid or | 125 |
| Δ¹-Cortienic acid methyl ester | |
| Lactose (anhydrous) | q.s. to 5000 |

Part (b) Composition

| Ingredients | μg per capsule |
|---|---|
| Zwitterion, e.g. (vii) [BOD-08] | 12-24 |
| Magnesium stearate | 0.15% to 0.25% by weight |
| Lactose (anhydrous) | q.s. to 5000 |

These inhalable powder compositions are formulated and administered as described in EXAMPLE 74.

### Example 82

Part (a) Composition

| Ingredients | μg per capsule |
|---|---|
| Ester compound of formula (1), e.g. | 50 |
| Compound 5, 8, 27, 31, or 35 | |
| Magnesium stearate | 0.15% to 0.25% by weight |
| Loteprednol etabonate | 125 |
| Δ¹-Cortienic acid or | 125 |
| Δ¹-Cortienic acid methyl ester | |
| Lactose (anhydrous) | q.s. to 5000 |

Part (b) Composition

| Ingredients | μg per capsule |
|---|---|
| Zwitterion, e.g. (vii) [BOD-08] | 10-20 |
| Magnesium stearate | 0.15% to 0.25% by weight |
| Lactose (anhydrous) | q.s. to 5000 |

These inhalable powder compositions are formulated and administered as described in EXAMPLE 38.

Yet other compositions can be conveniently formulated using known techniques.

Investigation of the anticholinergic action of ester compound and zwitterion combinations in acetylcholine induce bronchoconstruction in anesthetized guinea pigs

### Experimental Procedure

Male Hartley guinea pigs (320±120 g) (Charles River) are housed under standard conditions. Guinea pigs are anesthetized with urethane (2 g/kg, intraperitoneally), the trachea are cannulated and the animal is respired using a small animal respiratory pump (Harvard Apparatus LTD, Kent UK). Respiratory back pressure is measured and recorded using a rodent lung function recording system (MUMED, London UK). For drug administration the right jugular vein is cannulated. Following the surgical procedure, guinea pigs are allowed to stabilize for 20 minutes. Ten minutes before acetylcholine administration, the animals are disconnected from the ventilator and either the vehicle (10 mg lactose) or different amounts of the ester and zwitterion or their combination products (suspended in the same amount of vehicle) are administered intratracheally. The trachea is reconnected to the ventilator and changes in pulmonary mechanics are followed. Acetylcholine (10 μg/kg) is administered intravenously in every 10 minutes six times.

### Results

Compounds of formula (1) and (2) with zwitterions, their combination products and glycopyrrolate will exhibit a protective effect on the acetylcholine-induced bronchoconstriction provoked in this test.

This is a model for asthma, chronic obstructive pulmonary disorder and other obstructive respiratory tract disorders in which the effectiveness of the compounds of formulas (1) and (2) with zwitterions (i)-(x) and their combination products can be evaluated.

### Test for Bronchodilatory Effect of Inhaled Test Compounds in Balb/c Mice

Female BALB/c mice, weight range 19-22 g, are obtained, for example from Charles River Laboratories (Kingston, N.C.). They receive food and water ad libitum. [0176]Compounds for aerosol administration are prepared in sterile Dulbecco's Phosphate Buffered Saline. Mice are placed on a carousel-style, nose only, exposure chamber and allowed to inhale aerosols for five minutes, using an ICN SPAG-2 nebulizer. This nebulizer generates a mean aerosol particle size of 1.3 microns at a rate of approximately 0.25 ml/minute.

Ten minutes and 36 hours later, the mice are moved to whole bod plethysmograph chambers. Bronchoconstriction is induced in the mice by administration of an 80 mg/ml methacholine (MC) aerosol in the plethysmograph chambers for 5 minutes. The mice are allowed to inhale an aerosol containing 80 mg/ml methacholine following inhalation treatment with DPBS vehicle (Dulbecco's Phosphate Buffered Saline), or 80 mg/ml methacholine following inhalation treatment with test ester compound plus zwitterion or combination product. The average enhanced pause (Penh, lung resistance), corresponding to airflow resistance, is determined and statistically analyzed using Kruskal-Wallis one way ANOVA. In order to determine the baseline, saline aerosol (without methacholine) is also separately administered to the mice.

This procedure is a model for inhalation treatment of asthma, chronic obstructive pulmonary disorder and other obstructive respiratory tract disorders in which the ffectiveness of the compounds of formulas (1) or (2) together with zwitterions (i)-(x) or their combination products can be tested.

While this description has been couched in terms of various preferred or exemplary embodiments, the skilled artisan will appreciate that various modifications, substitutions, omissions and changes can be made without departing from the spirit thereof. Accordingly, it is intended that the scope of the foregoing be limited only by the broadest statements herein and by the scope of the following claims, including equivalents thereof.

1. (canceled)

2. A method of treating an obstructive disease of the respiratory tract in a subject suffering from same, the method comprising administering to the subject by oral inhalation or nasally, once or twice daily, at least one compound having the formula 2: 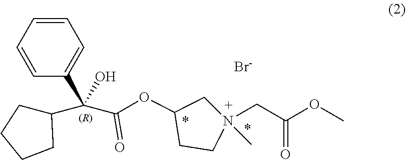
wherein R is C₁-C₈ straight or branched chain alkyl, said compound having the R stereoisomeric configuration at the 2 position and the R, S or RS stereoisomeric configuration at the 1′ and 3′ positions (designated by asterisks) or a stereoisomeric mixture thereof, in an amount of compound of formula (2) sufficient to reduce or inhibit at least one symptom of said obstructive disease with fewer systemic side-effects than glycopyrrolate.

3. (canceled)

4. A method of treating an obstructive disease of the respiratory tract in a subject suffering from same, said method comprising administering to the subject by oral inhalation or nasally, once or twice daily, (a) at least one ester compound having the formula: 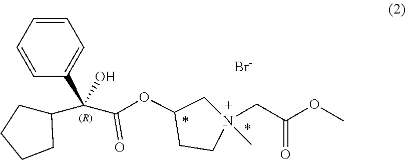
wherein R is C₁-C₈ straight or branched chain alkyl, said compound having the R stereoismeric configuration at the 2 position and the R, S or RS stereoisomeric configuration at the 1′ and 3′ positions (designated by asterisks) or a stereoisomeric mixture thereof, in an amount effective to reduce or inhibit at least one symptom of said obstructive disease with fewer systemic side-effects than glycopyrrolate; and (b) at least one zwitterion selected from the group consisting of:
(i) (±) 3-(2-cyclopentyl-2-phenyl-2-hydroxyacetoxy)-1-(carboxymethyl)-1-methylpyrrolidinium inner salt;
(ii) (2R) 3-(2-cyclopentyl-2-phenyl-2-hydroxyacetoxy)-1-(carboxymethyl)-1-methylpyrrolidinium inner salt;
(iii) (2R, 1′R, 3′R) 3-(2-cyclopentyl-2-phenyl-2-hydroxyacetoxy)-1-(carboxymethyl)-1-methylpyrrolidinium inner salt;
(iv) (2R, 1'S, 3′R) 3-(2-cyclopentyl-2-phenyl-2-hydroxyacetoxy)-1-(carboxymethyl)-1-methylpyrrolidinium inner salt;
(v) (2R, 1′R, 3'S) 3-(2-cyclopentyl-2-phenyl-2-hydroxyacetoxy)-1-(carboxymethyl)-1-methylpyrrolidinium inner salt;
(vi) (2R, 1'S, 3'S) 3-(2-cyclopentyl-2-phenyl-2-hydroxyacetoxy)-1-(carboxymethyl)-1-methylpyrrolidinium inner salt;
(vii) (2R, 3′R) 3-(2-cyclopentyl-2-phenyl-2-hydroxyacetoxy)-1-(carboxymethyl)-1-methylpyrrolidinium inner salt;
(viii) (2R, 3'S) 3-(2-cyclopentyl-2-phenyl-2-hydroxyacetoxy)-1-(carboxymethyl)-1-methylpyrrolidinium inner salt;
(ix) (2R, 1′R) 3-(2-cyclopentyl-2-phenyl-2-hydroxyacetoxy)-1-(carboxymethyl)-1-methylpyrrolidinium inner salt;
(x) (2R, 1'S) 3-(2-cyclopentyl-2-phenyl-2-hydroxyacetoxy)-1-(carboxymethyl)-1-methylpyrrolidinium inner salt; and
(xi) a mixture of said at least one zwitterion with at least one stereoisomer thereof,
in an amount or concentration sufficient to prolong the activity of the ester compound of formula (2) in reducing or inhibiting said at least one symptom.

5. The method of claim 2, wherein: (i) R is C₁-C₈ straight chain alkyl-; (ii); and/or (iii) the compound formula (2) and has the R or RS configuration at the 3′ position.

6-8. (canceled)

9. The method of claim 2, wherein the compound of formula (2) is selected from the group consisting of:
(a) (2R) 3-(2-cyclopentyl-2-phenyl-2-hydroxyacetoxy)-1-(methoxycarbonylmethyl)-1-methylpyrrolidinium bromide;
(b) (2R) 3-(2-cyclopentyl-2-phenyl-2-hydroxyacetoxy)-1-(ethoxycarbonylmethyl)-1-methylpyrrolidinium bromide;
(c) (2R) 3-(2-cyclopentyl-2-phenyl-2-hydroxyacetoxy-1-(n-hexyloxycarbonylmethyl)-1-methylpyrrolidinium bromide;
(d) (2R) 3-(2-cyclopentyl-2-phenyl-2-hydroxyacetoxy)-1-(n-octyloxycarbonylmethyl)-1-methylpyrrolidinium bromide;
(e) (2R) 3-(2-cyclopentyl-2-phenyl-2-hydroxyacetoxy)-1-(n-butoxycarbonylmethyl)-1-methylpyrrolidium bromide;
(f) (2R, 3′R) 3-(2-cyclopentyl-2-phenyl-2-hydroxyacetoxy)-1-(methoxycarbonylmethyl)-1-methylpyrrolidinium bromide;
(g) (2R, 3′R) 3-(2-cyclopentyl-2-phenyl-2-hydroxyacetoxy)-1-(ethoxycarbonylmethyl)-1-methylpyrrolidinium bromide;
(h) (2R, 3′R) 3-(2-cyclopentyl-2-phenyl-2-hydroxyacetoxy)-1-(n-hexyloxycarbonylmethyl)-1-methylpyrrolidium bromide;
(i) (2R, 3′R) 3-(2-cyclopentyl-2-phenyl-2-hydroxyacetoxy)-1-(n-octyloxycarbonylmethyl)-1-methylpyrrolidinium bromide; and
(j) (2R, 3′R) 3-(2-cyclopentyl-2-phenyl-2-hydroxyacetoxy)-1-(n-butoxycarbonylmethyl)-1-methylpyrrolidinium bromide.

10. The method of claim 2, wherein the compound of formula (2) is selected from the group consisting of:
(a) (2R) 3-(2-cyclopentyl-2-phenyl-2-hydroxyacetoxy)-1-(methoxycarbonylmethyl)-1-methylpyrrolidinium bromide;
(b) (2R) 3-(2-cyclopentyl-2-phenyl-2-hydroxyacetoxy)-1-(ethoxycarbonylmethyl)-1-methylpyrrolidinium bromide;
(f) (2R, 3′R) 3-(2-cyclopentyl-2-phenyl-2-hydroxyacetoxy)-1-(methoxycarbonylmethyl)-1-methylpyrrolidinium bromide; and
(g) (2R, 3′R) 3-(2-cyclopentyl-2-phenyl-2-hydroxyacetoxy)-1-(ethoxycarbonylmethyl)-1-methylpyrrolidinium bromide.

11-12. (canceled)

13. The method of claim 4, wherein the zwitterion is selected from the group consisting of:
(ii) (2R) 3-(2-cyclopentyl-2-phenyl-2-hydroxyacetoxy)-1-(carboxymethyl)-1-methylpyrrolidinium inner salt; and
(vii) (2R, 3′R) 3-(2-cyclopentyl-2-phenyl-2-hydroxyacetoxy)-1-(carboxymethyl)-1-methylpyrrolidinium inner salt.

14-16. (canceled)

17. The method of claim 2, wherein the compound of formula (2) is administered in an amount of (a) from about 20 μg to about 150 μg per dose or (b) from about 50 μg to about 100 μg per dose and is administered by oral inhalation.

18-20. (canceled)

21. The method of claim 4, wherein the ester compound of formula (2) is administered in an amount of from about 20 μg to about 150 μg per dose and wherein the ester compound and the zwitterion are administered by oral inhalation.

22. (canceled)

23. The method of claim 2, wherein the obstructive disease of the respiratory tract is selected from the group consisting of asthma, bronchitis, chronic obstructive pulmonary disease (COPD), allergic rhinitis, infectious rhinitis, bronchiectasis, acute lung injury (ALI), acute respiratory distress syndrome (ARDS) and cystic fibrosis.

24-26. (canceled)

27. The method of claim 3, wherein the obstructive disease of the respiratory tract is selected from the group consisting of asthma, bronchitis, chronic obstructive pulmonary disease (COPD), allergic rhinitis, infectious rhinitis, bronchiectasis, acute lung injury (ALI), acute respiratory distress syndrome (ARDS) and cystic fibrosis.

28-69. (canceled)

70. The method of claim 2, wherein said ester compound is administered in the form of a pharmaceutical composition comprising (a) said ester compound, and (b) a non-toxic pharmaceutically acceptable carrier therefor.

71. The method of claim 4, wherein said ester compound and, optionally, said zwitterion are administered in the form of a pharmaceutical composition comprising (a) said ester compound, (b) a non-toxic pharmaceutically acceptable carrier therefor, and, optionally, (c) a zwitterion, formulated for administration by oral inhalation.

72. The method of claim 71, wherein the composition is formulated as an inhalable powder.

73. (canceled)

74. A pharmaceutical composition comprising: (a) at least one ester compound having the formula 2: 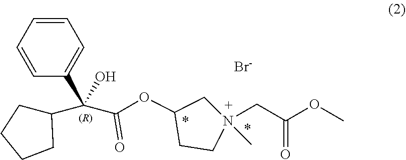
wherein R is C₁-C₈ straight or branched chain alkyl, said compound having the R stereoismeric configuration at the 2 position and the R, S or RS stereoisomeric configuration at the 1′ and 3′ positions (designated by asterisks) or a stereoisomeric mixture thereof, in an amount effective to reduce or inhibit at least one symptom of said obstructive disease with fewer systemic side-effects than glycopyrrolate; and (b) at least one zwitterion selected from the group consisting of:
(i) (±) 3-(2-cyclopentyl-2-phenyl-2-hydroxyacetoxy)-1-(carboxymethyl)-1-methylpyrrolidinium inner salt;
(ii) (2R) 3-(2-cyclopentyl-2-phenyl-2-hydroxyacetoxy)-1-(carboxymethyl)-1-methylpyrrolidinium inner salt;
(iii) (2R, 1′R, 3′R) 3-(2-cyclopentyl-2-phenyl-2-hydroxyacetoxy)-1-(carboxymethyl)-1-methylpyrrolidinium inner salt;
(iv) (2R, 1'S, 3′R) 3-(2-cyclopentyl-2-phenyl-2-hydroxyacetoxy)-1-(carboxymethyl)-1-methylpyrrolidinium inner salt;
(v) (2R, 1′R, 3'S) 3-(2-cyclopentyl-2-phenyl-2-hydroxyacetoxy)-1-(carboxymethyl)-1-methylpyrrolidinium inner salt;
(vi) (2R, 1'S, 3'S) 3-(2-cyclopentyl-2-phenyl-2-hydroxyacetoxy)-1-(carboxymethyl)-1-methylpyrrolidinium inner salt;
(vii) (2R, 3′R) 3-(2-cyclopentyl-2-phenyl-2-hydroxyacetoxy)-1-(carboxymethyl)-1-methylpyrrolidinium inner salt;
(viii) (2R, 3'S) 3-(2-cyclopentyl-2-phenyl-2-hydroxyacetoxy)-1-(carboxymethyl)-1-methylpyrrolidinium inner salt;
(ix) (2R, 1′R) 3-(2-cyclopentyl-2-phenyl-2-hydroxyacetoxy)-1-(carboxymethyl)-1-methylpyrrolidinium inner salt;
(x) (2R, 1'S) 3-(2-cyclopentyl-2-phenyl-2-hydroxyacetoxy)-1-(carboxymethyl)-1-methylpyrrolidinium inner salt; and
(xi) a mixture of said at least one zwitterion with at least one stereoisomer thereof,
in an amount or concentration sufficient to prolong the activity of the ester compound of formula (2) in reducing or inhibiting said at least one symptom; and (c) a non-toxic pharmaceutically acceptable carrier therefor.

75-78. (canceled)

79. The composition of claim 74, wherein the ester compound of formula (2) is selected from the group consisting of:
(a) (2R) 3-(2-cyclopentyl-2-phenyl-2-hydroxyacetoxy)-1-(methoxycarbonylmethyl)-1-methylpyrrolidinium bromide;
(b) (2R) 3-(2-cyclopentyl-2-phenyl-2-hydroxyacetoxy)-1-(ethoxycarbonylmethyl)-1-methylpyrrolidinium bromide;
(c) (2R) 3-(2-cyclopentyl-2-phenyl-2-hydroxyacetoxy-1-(n-hexyloxycarbonylmethyl)-1-methylpyrrolidinium bromide;
(d) (2R) 3-(2-cyclopentyl-2-phenyl-2-hydroxyacetoxy)-1-(n-octyloxycarbonylmethyl)-1-methylpyrrolidinium bromide;
(e) (2R) 3-(2-cyclopentyl-2-phenyl-2-hydroxyacetoxy)-1-(n-butoxycarbonylmethyl)-1-methylpyrrolidium bromide;
(f) (2R, 3′R) 3-(2-cyclopentyl-2-phenyl-2-hydroxyacetoxy)-1-(methoxycarbonylmethyl)-1-methylpyrrolidinium bromide;
(g) (2R, 3′R) 3-(2-cyclopentyl-2-phenyl-2-hydroxyacetoxy)-1-(ethoxycarbonylmethyl)-1-methylpyrrolidinium bromide;
(h) (2R, 3′R) 3-(2-cyclopentyl-2-phenyl-2-hydroxyacetoxy)-1-(n-hexyloxycarbonylmethyl)-1-methylpyrrolidium bromide;
(i) (2R, 3′R) 3-(2-cyclopentyl-2-phenyl-2-hydroxyacetoxy)-1-(n-octyloxycarbonylmethyl)-1-methylpyrrolidinium bromide; and
(j) (2R, 3′R) 3-(2-cyclopentyl-2-phenyl-2-hydroxyacetoxy)-1-(n-butoxycarbonylmethyl)-1-methylpyrrolidinium bromide.

80. The composition of claim 79, wherein the ester compound of formula (2) is selected from the group consisting of:
(a) (2R) 3-(2-cyclopentyl-2-phenyl-2-hydroxyacetoxy)-1-(methoxycarbonylmethyl)-1-methylpyrrolidinium bromide;
(b) (2R) 3-(2-cyclopentyl-2-phenyl-2-hydroxyacetoxy)-1-(ethoxycarbonylmethyl)-1-methylpyrrolidinium bromide;
(f) (2R, 3′R) 3-(2-cyclopentyl-2-phenyl-2-hydroxyacetoxy)-1-(methoxycarbonylmethyl)-1-methylpyrrolidinium bromide; and
(g) (2R, 3′R) 3-(2-cyclopentyl-2-phenyl-2-hydroxyacetoxy)-1-(ethoxycarbonylmethyl)-1-methylpyrrolidinium bromide.

81-84. (canceled)

85. The composition of claim 80, wherein the zwitterion is selected from the group consisting of:
(ii) (2R) 3-(2-cyclopentyl-2-phenyl-2-hydroxyacetoxy)-1-(carboxymethyl)-1-methylpyrrolidinium inner salt; and
(vii) (2R, 3′R) 3-(2-cyclopentyl-2-phenyl-2-hydroxyacetoxy)-1-(carboxymethyl)-1-methylpyrrolidinium inner salt.

86. (canceled)

87. The composition of claim 74, wherein the ester compound of formula (2) is present in an amount selected from the group consisting of (a) from about 20 μg to about 150 μg per dose or (b) from about 50 μg to about 100 μg per dose and wherein the composition is formulated for oral inhalation.

88. (canceled)

89. The method of claim 4, wherein the amount of said at least one zwitterion is insufficient alone to reduce or inhibit at least one symptom of said obstructive disease; and/or wherein the ester compound and the zwitterion are present in a molar or weight ratio of ester compound:zwitterion of from about 2.5:1 to about 5:1.

90. (canceled)

91. The composition of claim 74, wherein the amount of said at least one zwitterion is insufficient alone to reduce or inhibit at least one symptom of said obstructive disease; and/or wherein the ester compound and the zwitterion are present in a molar or weight ratio of ester compound:zwitterion of from about 2.5:1 to about 5:1.

92. (canceled)

93. The method of claim 4, wherein said ester compound and, optionally, said zwitterion are administered to said subject as a pharmaceutical combination further comprising an anti-inflammatory corticosteroid, a betamimetic agent or an antiallergic agent, and wherein the combined amount of ester compound and anti-inflammatory corticosteroid, betamimetic agent or antiallergic agent is effective to reduce or inhibit at least one symptom of said obstructive disease of the respiratory tract.

94. The method of claim 93, wherein: the pharmaceutical combination further comprises a non-toxic pharmaceutically acceptable carrier ii the combination is formulated as an inhalable powder; (iii) the anti-inflammatory corticosteroid is selected from the group consisting of bunesonide, fluticasone, loteprednol etabonate, etiprednol dichloracetate, mometasone and ciclesonide; and/or (iv) the betamimetic agent is selected from the group consisting of fenoterol, formoterol and salmeterol.

95-97. (canceled)

98. The method of claim 94, wherein the anti-inflammatory steroid is loteprednol etabonate and wherein the pharmaceutical combination further comprises an enhancing agent for the loteprednol etabonate selected from the group consisting of:
(a) 11β,17α-dihydroxyandrost-4-en-3-one-17β-carboxylic acid;
(b) 11β,17α-dihydroxyandrosta-1,4-dien-3-one-17β-carboxylic acid;
(c) methyl 11β,17α-dihydroxyandrost-4-en-3-one-17β-carboxylate;
(d) ethyl 11β, 17α-dihydroxyandrost-4-en-3-one-17β-carboxylate;
(e) methyl 11β,17α-dihydroxyandrosta-1,4-dien-3-one-17β-carboxylate; and
(f) ethyl 11β,17α-dihydroxyandrosta-1,4-dien-3-one-17β-carboxylate.

99. The composition of claim 74, wherein the ester compound and the zwitterion are present in a pharmaceutical combination further comprising an anti-inflammatory corticosteroid, a betamimetic agent or an antiallergic agent, and wherein the combined amount of ester compound and anti-inflammatory corticosteroid, betamimetic agent or antiallergic agent is effective to reduce or inhibit at least one symptom of said obstructive disease of the respiratory tract.

100. The composition of claim 99, wherein: (i) the pharmaceutical combination further comprises a non-toxic pharmaceutically acceptable carrier; (ii) the combination is formulated as an inhalable powder; (iii) the anti-inflammatory corticosteroid is selected from the group consisting of bunesonide, fluticasone, loteprednol etabonate, etiprednol dichloracetate, mometasone and ciclesonide; and/or (iv) the betamimetic agent is selected from the group consisting of fenoterol, formoterol and salmeterol.

101-103. (canceled)

104. The composition of claim 100, wherein the anti-inflammatory steroid is loteprednol etabonate and wherein the pharmaceutical combination further comprises an enhancing agent for the loteprednol etabonate selected from the group consisting of:
(a) 11β,17α-dihydroxyandrost-4-en-3-one-17β-carboxylic acid;
(b) 11β, 17α-dihydroxyandrosta-1,4-dien-3-one-17β-carboxylic acid;
(c) methyl 11β,17α-dihydroxyandrost-4-en-3-one-17β-carboxylate;
(d) ethyl 11β, 17α-dihydroxyandrost-4-en-3-one-17β-carboxylate;
(e) methyl 11β,17α-dihydroxyandrosta-1,4-dien-3-one-17β-carboxylate; and
(f) ethyl 11β,17α-dihydroxyandrosta-1,4-dien-3-one-17β-carboxylate.
